(19) United States
(12) Patent Application Publication
(10) Pub. No.: US 2015/0045740 A1
(43) Pub. Date: Feb. 12, 2015

(54) SYRINGE AND MOUNTING FIXTURE

(71) Applicant: TERUMO KABUSHIKI KAISHA, Shibuya-ku (JP)

(72) Inventors: Junya KOJIMA, Ashigarakami-gun (JP); Shinichi Kaneda, Ashigarakami-gun (JP); Yasuo Kurosaki, Ashigarakami-gun (JP)

(73) Assignee: TERUMO KABUSHIKI KAISHA, Tokyo (JP)

(21) Appl. No.: 14/520,970

(22) Filed: Oct. 22, 2014

Related U.S. Application Data
(63) Continuation of application No. PCT/JP2013/057379, filed on Mar. 15, 2013.

(30) Foreign Application Priority Data
Apr. 24, 2012 (JP) 2012-098862

(51) Int. Cl. A61M 5/315

(52) U.S. Cl. CPC A61M 5/31505; A61M 2005/31508; A61M 2005/3151
USPC 604/220

(57) ABSTRACT

Provided are a syringe and a mounting fixture by which it can be facilitated to perform delivery of a target amount of a liquid selectively only when necessary. A syringe is provided with: an external cylinder which is fillable with a liquid; a plunger which is translatable within the external cylinder along the longitudinal direction of the external cylinder and which is formed with a plurality of concave portions and convex portions aligned in the longitudinal direction; and an annular mounting fixture which is mountable to the external cylinder. The mounting fixture is provided with: protruding portions which contact at least the convex portions sequentially in a slidable manner as the plunger is translated; and a rotation limiting member which contacts the plunger to limit a rotation of the plunger relative to the external cylinder.

### CROSS-REFERENCES TO RELATED APPLICATIONS

This application is a continuation of International Application No. PCT/JP2013/057379 filed on Mar. 15, 2013, and claims priority to Japanese Application No. 2012-098862 filed on Apr. 24, 2012, the entire content of each of which are incorporated herein by reference.

### TECHNICAL FIELD

The present invention relates to a syringe operable to discharge a liquid filling an external cylinder by translation of a plunger, and a mounting fixture configured to be mounted to the syringe.

### BACKGROUND DISCUSSION

In the medical field, syringes are commonly used as an instrument for administering a liquid such as a medicinal liquid into an affected part. In using a syringe, a plunger inside an external cylinder is translated rearward (proximally) to suck a liquid into the external cylinder via a flow port at the forward end (distal end) of the external cylinder to fill the external cylinder, and the plunger is translated toward the forward end of the external cylinder, whereby the liquid can be delivered through the flow port.

The external cylinder of such a syringe is usually provided thereon with graduations at regular intervals along the translation direction of the plunger. By pushing the plunger while checking the displacement of the plunger through observation of the graduations, a predetermined amount of liquid can be delivered. It can be difficult, however, to stop the plunger at a predetermined graduation, without extensive training or experience. For example, it is necessary to control the force applied to the plunger, according to the viscosity of the liquid, the diameters of the plunger and the external cylinder, the object into which the liquid is to be delivered, etc.

In view of this, for making it possible to control the force applied to the plunger through facilitated grasping of the amount of liquid delivered, for example, Japanese Patent Laid-Open No. 2009-28527 discloses a syringe configured such that the amount of a liquid delivered can be determined through a sensation of a change in resistance at the time of pushing a plunger, without the need for checking of graduations. The syringe described in Japanese Patent Laid-Open No. 2009-28527 has a configuration in which a projecting portion is formed on the inside of a rear-side opening portion of the external cylinder, and a plurality of contact portions aligned at a predetermined pitch are formed on the outside of the plunger so that the contact portions sequentially make contact with the projecting portion as the plunger is pushed in. When the plunger is pushed into the external cylinder, therefore, the contact portions of the plunger sequentially make contact with the projecting portion of the external cylinder. Accordingly, the operator can recognize the amount of the liquid delivered, by detecting a variation in the pushing-in resistance due to the contact by way of a sensation in the finger.

### SUMMARY

The syringe described above, however, has the following problem. Since both the contact portions and the projecting portion are formed on the plunger and the external cylinder, the contact portions necessarily contact the projecting portion, even when sucking the liquid into the external cylinder, or in the cases where detection of the sensation of a variation in resistance is not needed. Accordingly, it is an object of the present disclosure to provide a syringe and a mounting fixture by which performance of selective delivery of a target amount of a liquid is facilitated only when necessary.

The above object is achieved by a syringe including: an external cylinder which is fillable with a liquid; a plunger which is translatable within the external cylinder along a longitudinal direction of the external cylinder, the plunger formed with a plurality of concave portions and convex portions aligned in the longitudinal direction; and a mounting fixture that includes a ring-shaped annular member mountable to the external cylinder, a protruding portion configured to, upon translation of the plunger within the external cylinder, contact at least the plurality of convex portions sequentially in a slidable manner or stop movement of the convex portions, and a rotation limiting member contacting the plunger so as to limit a rotation of the plunger relative to the external cylinder.

According to the syringe and the mounting fixture configured as above, the mounting fixture is in a ring shape. This ensures that the mounting fixture can be mounted to the external cylinder so as to surround the external cylinder, and the mounting fixture can be mounted selectively only when necessary. In addition, the mounting fixture has the protruding portion and the rotation limiting member. Notwithstanding the mounting fixture is of a selectively mounted type, therefore, rotation of the plunger is limited by the rotation limiting member, so that the contact of the protruding portion with the convex portions is secured reliably. Accordingly, the protruding portion contacts the convex portions sequentially, and resisting forces exerted on the plunger from the protruding portion are transmitted to the finger assuredly. Consequently, the amount of the liquid delivered can be recognized through a sensation in the finger, and delivery of a target amount of the liquid is facilitated.

The annular member may be formed in a discontinuous ring shape mountable to the external cylinder by being mounted on the external cylinder or the plunger from a direction crossing the longitudinal direction of the external cylinder. This configuration makes it possible to mount the annular member to the external cylinder in the manner of mounting the annular member on the external cylinder or the plunger from a direction crossing the longitudinal direction of the external cylinder. Consequently, the mounting fixture can be mounted selectively only when necessary.

The mounting fixture may have a first interlock member and a second interlock member interlockable to each other and located at respective discontinuous ends. This configuration ensures that the mounting fixture is fixed to the external cylinder assuredly, and a stable liquid delivery operation can be achieved.

The annular member may have a deformable portion which is lower in flexural rigidity than other portions of the annular member and which can be deformed so as to bring the first interlock member and the second interlock member closer to and away from each other. This configuration ensures that the discontinuous parts of the mounting fixture can be easily opened in order to dispose the external cylinder and the plunger into the inside of the mounting fixture. As a result, the mounting is facilitated, and operability is enhanced.

The mounting fixture may be so configured that two discontinuous ends are located apart from each other in a state in which the mounting fixture is mounted to the external cylinder. This configuration facilitates insertion of the external cylinder and the plunger into the inside of the mounting fixture, and enhances operability. In addition, since both discontinuous end portions are located apart from each other and need not be interlocked, upon insertion of differently sized external cylinder and plunger the mounting fixture can deform so as to open the discontinuous portions wider according to the shapes of the external cylinder and plunger. Therefore, it is possible to cope with a variety of external cylinders and plungers while using a single mounting fixture.

The rotation limiting member may have one or more pairs of guide members configured so as to clip the plunger from a direction perpendicular to the longitudinal direction of the external cylinder. This makes it possible to limit the rotation of the plunger more reliably, and to perform a stable liquid delivery operation.

The one or more pairs of guide members may be closely aligned so as to clip the plunger, from a spaced relationship with each other, through deformation of the mounting fixture when the mounting fixture is mounted. This ensures that it is easy to dispose the plunger between the spaced apart guide members, and, after the mounting, rotation of the plunger can be limited reliably by the closely aligned guide members.

The mounting fixture may have a flange-engaging member engaging a flange at an edge of an opening portion of the external cylinder in which the plunger is inserted. This configuration ensures that the mounting fixture can resist forces exerted thereon from the plunger due to forward and rearward translations of the plunger. Consequently, the mounting fixture would not easily be disengaged from the external cylinder, and a stable liquid delivery operation can be achieved.

The mounting fixture may include: a rear-side main body disposed in a side of the opening portion of the flange of the external cylinder; a forward-side main body disposed opposite the side of the opening portion of the flange; a connecting member connecting the forward-side main body and the rear-side main body; and a main body engaging member capable of interlocking the forward-side main body and the rear-side main body. In this configuration, with the flange clipped by the forward-side main body and the rear-side main body and with the forward-side main body and the rear-side main body interlocked together by the main body engaging member, the mounting fixture is fixed to the external cylinder assuredly, and a stable liquid delivery operation can be achieved.

The connecting member may have a connecting member deformable portion which is lower in flexural rigidity than other portions of the connecting member and which is deformable so as to bring the forward-side main body and the rear-side main body closer to and away from each other. This ensures that the forward-side main body and the rear-side main body can be opened so as to be spaced from each other. Accordingly, the mounting of the mounting fixture is facilitated, and operability is enhanced.

The annular member may be a flat-shaped member in a flat plate form that is mountable to the external cylinder by deformation into a ring shape. This configuration ensures that by deforming the flat-shaped member into a ring shape it is possible to mount the mounting fixture selectively only when necessary.

The flat-shaped member may have a deformable portion or portions lower in flexural rigidity than other portions of the flat-shaped member so that the flat-shaped member can be deformed into a ring shape. This configuration enables the flat-shaped member to be easily deformed into a ring shape. Accordingly, mounting of the mounting fixture is facilitated, and operability is enhanced.

The annular member may be formed in a ring shape mountable to the external cylinder by inserting the plunger therein from a rear end side of the plunger. This makes it possible to mount the mounting fixture to the external cylinder from the rear end side of the plunger, and to mount the mounting fixture selectively only when necessary.

The annular member may include: a pair of pressing members located opposite each other; and a pair of translating members formed between the pair of pressing members and translating so as to be spaced from each other by pressing the pair of pressing members closer to each other. This configuration ensures that the mounting fixture can be mounted from the rear end side of the plunger, with the pair of translating members put in a spaced relationship with each other by pressing the pressing members. Accordingly, the mounting is facilitated, and operability is enhanced.

The protruding portion may be formed to extend from the translating member toward the plunger. This configuration enables the protruding portion to be spaced apart from the plunger together with the translating member at the time of mounting the mounting fixture. As a result, the mounting is facilitated and operability is enhanced.

The rotation limiting member may be formed to extend from the translating member toward the plunger. This permits the rotation limiting member to be spaced away from the plunger together with the translating member at the time of mounting the mounting fixture. Accordingly, the mounting is facilitated and operability is enhanced.

The mounting fixture may have a flange-engaging member engaging a flange at an edge of an opening portion of the external cylinder in which the plunger is inserted. This configuration ensures that the mounting fixture can resist forces exerted thereon from the plunger due to forward and rearward translations of the plunger. Consequently, the mounting fixture would not easily be disengaged from the external cylinder, and a stable liquid delivery operation can be achieved.

The annular member may have a deformable portion lower in flexural rigidity than other portions of the annular member. This configuration enables the annular member to be easily deformed so as to allow easy insertion of the plunger therein. As a result, operability is enhanced.

The protruding portion may be a protruding stopper that stops a translation of the plunger by contacting the plunger as the plunger translates. This configuration ensures that, notwithstanding the mounting fixture is of a selectively mounted type, rotation of the plunger is limited by the rotation limiting member, and the contact of the protruding stopper with the plunger is secured reliably. Accordingly, delivery of a liquid in an amount more than intended can be securely prevented by the protruding stopper that stops the plunger. Consequently, delivery of a target amount of liquid can be performed easily.

A configuration may be adopted wherein: the convex portion is a convex stopper that is capable of contacting the protruding stopper; the mounting fixture has a pressing member configured to fix the protruding stopper; and the protruding stopper cancels holding the plunger by moving in a circumferential direction of the plunger from a position of contact with the convex stopper when the pressing member is pressed. This configuration ensures that holding the plunger can be easily canceled by an operation of pressing the pressing member, which enhances operability.

The mounting fixture may have a pressing member support which is adjacent to the pressing member and is lower in flexural rigidity than the pressing member. This facilitates pressing-in of the pressing member, thereby enhancing operability.

The mounting fixture may have a flange-engaging member engaging a flange at an edge of an opening portion of the external cylinder in which the plunger is inserted. This configuration ensures that the mounting fixture can resist forces exerted thereon from the plunger due to forward and rearward translations of the plunger. Consequently, the mounting fixture would not easily be disengaged from the external cylinder, and a stable liquid delivery operation can be achieved.

A configuration may be adopted wherein the plunger has a plurality of concave and convex portion-forming members each of which has the plurality of concave portions and convex portions aligned in the longitudinal direction of the plunger and which are aligned about an axis line of the plunger, and the pitch of the concave portions and convex portions differs depending on the concave and convex portion-forming members. This configuration ensures that the concave and convex portion-forming member to be brought into contact with the protruding portion can be selected from a plurality of ones, and the unit amount in which the liquid can be delivered can be selected from a plurality of choices.

### BRIEF DESCRIPTION OF DRAWINGS

FIG. 1 is a perspective view showing a syringe according to a first embodiment.

FIG. 2 is a plan view showing an external cylinder of the syringe according to the first embodiment.

FIG. 3 illustrates a plunger of the syringe according to the first embodiment, wherein FIG. 3(A) is a plan view and FIG. 3(B) is a view along arrow A in FIG. 3(A).

FIG. 4 is a perspective view showing a natural state of a mounting fixture of the syringe according to the first embodiment.

FIG. 5 is a plan view showing the natural state of the mounting fixture of the syringe according to the first embodiment.

FIG. 6 is a plan view showing a mounted state of the mounting fixture of the syringe according to the first embodiment.

FIG. 7 shows sectional views showing how a plunger is pressed after mounting the mounting fixture of the syringe according to the first embodiment, wherein FIG. 7(A) shows a state when a protruding portion contacts a concave portion, whereas FIG. 7(B) shows a state when the protruding portion contacts a convex portion.

FIG. 8 is a plan view showing a modification of the mounting fixture of the syringe according to the first embodiment.

FIG. 9 is a plan view showing a mounting fixture of a syringe according to a second embodiment.

FIG. 10 is a view showing the mounting fixture, as viewed along arrow B in FIG. 9.

FIG. 11 is a view showing the mounting fixture, as viewed along arrow C in FIG. 9.

FIG. 12 is a plan view showing a state when the mounting fixture in the second embodiment is mounted.

FIG. 13 shows sectional views showing how a plunger is pressed after mounting of the mounting fixture of the syringe according to the second embodiment, wherein FIG. 13(A) shows a state when a protruding portion contacts a concave portion, whereas FIG. 13(B) shows a state when the protruding portion contacts a convex portion.

FIG. 14 is a plan view showing a modification of the mounting fixture of the syringe according to the second embodiment.

FIG. 15 is a plan view showing a mounting fixture of a syringe according to a third embodiment.

FIG. 16 illustrates an unfolded state of the mounting fixture in the third embodiment, wherein FIG. 16(A) is a plan view, and FIG. 16(B) is a view along arrow D in FIG. 16(A).

FIG. 17 shows plan views of a first interlock portion and a second interlock portion of the mounting fixture in the third embodiment, wherein FIG. 17(A) shows a state in the course of interlocking, and FIG. 17(B) shows a state after the interlocking.

FIG. 18 shows plan views showing how a plunger is pressed after mounting of the mounting fixture of the syringe according to the third embodiment, wherein FIG. 18(A) shows a state when a protruding portion contacts a concave portion, whereas FIG. 18(B) shows a state when the protruding portion contacts a convex portion.

FIG. 19 is a plan view showing a mounting fixture of a syringe according to a fourth embodiment.

FIG. 20 is a view of the mounting fixture, as viewed along arrow E in FIG. 19.

FIG. 21 is a plan view showing a pressed state of a pressing portion of the mounting fixture of the syringe according to the fourth embodiment.

FIG. 22 shows plan views showing how the mounting fixture in the fourth embodiment engages an external cylinder, wherein FIG. 22(A) shows a state before engagement, and FIG. 22(B) shows a state after the engagement.

FIG. 23 shows plan views showing how a plunger is pressed after mounting of the mounting fixture of the syringe according to the fourth embodiment, wherein FIG. 23(A) shows a state when a protruding portion contacts a concave portion, whereas FIG. 23(B) shows a state when the protruding portion contacts a convex portion.

FIG. 24 is a plan view showing a natural state of a mounting fixture in a fifth embodiment.

FIG. 25 is a view of the mounting fixture, as viewed along arrow F in FIG. 24.

FIG. 26 illustrates a plunger in the fifth embodiment, wherein FIG. 26(A) is a plan view, and FIG. 26(B) is a view along arrow G in FIG. 26(A).

FIG. 27 is a plan view showing a mounted state of the mounting fixture of the syringe according to the fifth embodiment.

FIG. 28 shows sectional views showing how a plunger is pressed after mounting of the mounting fixture of the syringe according to the fifth embodiment, wherein FIG. 28(A) shows a state before the plunger is stopped, whereas FIG. 28(B) shows a state when the plunger is stopped.

FIG. 29 is a plan view of the mounting fixture, for illustrating how the holding of the plunger in the fifth embodiment is canceled.

FIG. 30 is a sectional view of the syringe, for illustrating how the holding of the plunger in the fifth embodiment is canceled.

### DETAILED DESCRIPTION

Now, embodiments of the syringe and mounting fixture will be described below, referring to the drawings. It is to be noted that the dimensional ratios in the drawings may be exaggerated and be different from the actual ratios, for convenience of description.

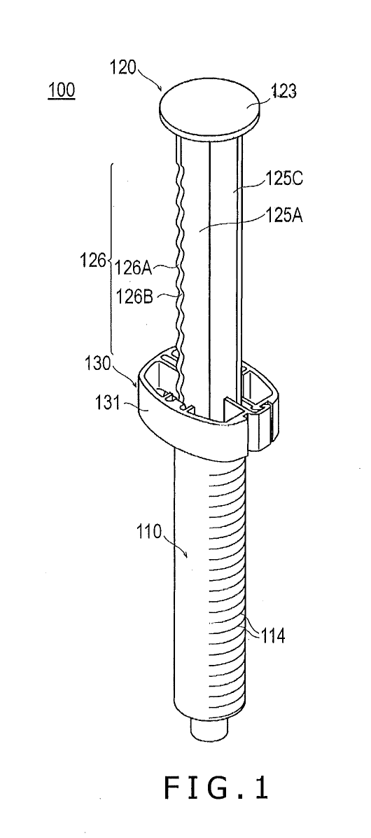

As shown in FIG. 1, a syringe 100 according to a first embodiment includes: an external cylinder 110; a plunger 120 translatable within the external cylinder 110 along the longitudinal direction of the external cylinder 110; and a mounting fixture 130 mountable to the external cylinder 110.

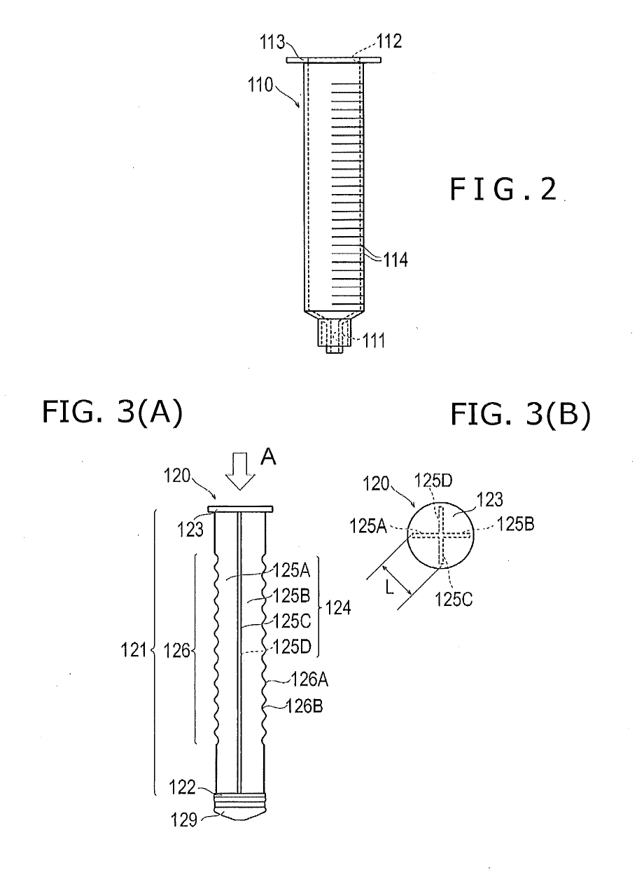

As shown in FIGS. 1 and 2, the external cylinder 110 is in a hollow roughly cylindrical form. The external cylinder 110 is formed on a forward side (distal side) thereof with a flow port 111 for sucking a liquid into the external cylinder 110 or discharging a liquid out of the external cylinder 110, and is formed on a rear side (proximal side) thereof with an insertion port 112 through which the plunger 120 is insertable. It is to be noted that herein the side on which the flow port 111 of the external cylinder 110 is provided is referred to as “forward (end)” or “forward side,” and the operator's side on which the plunger 120 is provided and operated is referred to as “rear (end)” or “rear side.”

At an edge of the insertion port 112 of the external cylinder 110 is formed a flange 113 projecting in directions perpendicular to the longitudinal direction of the external cylinder 110. The flange 113 is formed in a roughly rectangular shape elongated in one direction so that operator's fingers can be easily put thereon. The external cylinder 110 is provided on an external surface thereof with graduations 114 at regular intervals along the longitudinal direction of the external cylinder 110 so that the distance the plunger 120 inside is translated can be visually checked. The external cylinder 110 is not specifically restricted in regard of material; preferably, however, the material is a resin such as, for example, polyvinyl chloride, polyethylene, polypropylene, cyclic polyolefin, polystyrene, poly(4-methylpentene-1), polycarbonate, acrylic resin, acrylonitrile-butadiene-styrene copolymer, polyesters such as polyethylene terephthalate, polyethylene naphthalate, etc., butadiene-styrene copolymer, polyamides (e.g., nylon 6, nylon 6.6, nylon 6.10, nylon 12), etc. It is to be noted that the external cylinder 110 is preferably transparent or semi-transparent for securing visibility of the inside thereof, but the external cylinder 110 may be opaque.

As shown in FIGS. 1 and 3, the plunger 120 is provided with: a plunger main body 121 extending in the longitudinal direction of the external cylinder 110; and a gasket 129 interlocked to the forward side of the plunger main body 121. The plunger main body 121 includes: a plunger forward end 122 provided on the forward side and configured to interlock with the gasket 129; an operating member 123 provided on the rear side and capable of being pushed or pulled by operator's fingers; and a plunger extending member 124 extending long from the operating member 123 to the plunger forward end 124. The plunger extending member 124 has four plate form blade members 125A to 125D aligned about the axis of the external cylinder 110.

Of the four blade members 125A to 125D, the two blade members 125A and 125B (concave and convex portion-forming members) located on opposite sides of the axis of the external cylinder 110 are each formed with a concave and convex portion 126. The concave and convex portion 126 has repeating structures aligned at regular intervals (at a regular pitch), each of the repeating structures composed of a convex portion 126A and a concave portion 126B. The form of the concave and convex portion 126 is not specifically restricted insofar as it has concave portions 126B and convex portions 126A formed in a repeated manner. Examples of the form of the concave and convex portion 126 which can be adopted include a wavy form in which convex portions and concave portions are formed in a continuous manner, a form in which convex portions are periodically formed on a straight shaped portion (in this case, the straight portion corresponds to concave portions), and a form in which concave portions are periodically formed in a straight shaped portion (in this case, the straight portion corresponds to convex portions).

The other two blade members 125C and 125D are not particularly formed with such shapes as concave and convex portions, but are formed to be straight in the longitudinal direction. It is to be noted that the number of the blade members 125A to 125D of the plunger 120 may not necessarily be four. The material of the plunger main body 121 is not specifically restricted; for example, the aforementioned materials applicable to the external cylinder 110 can be used.

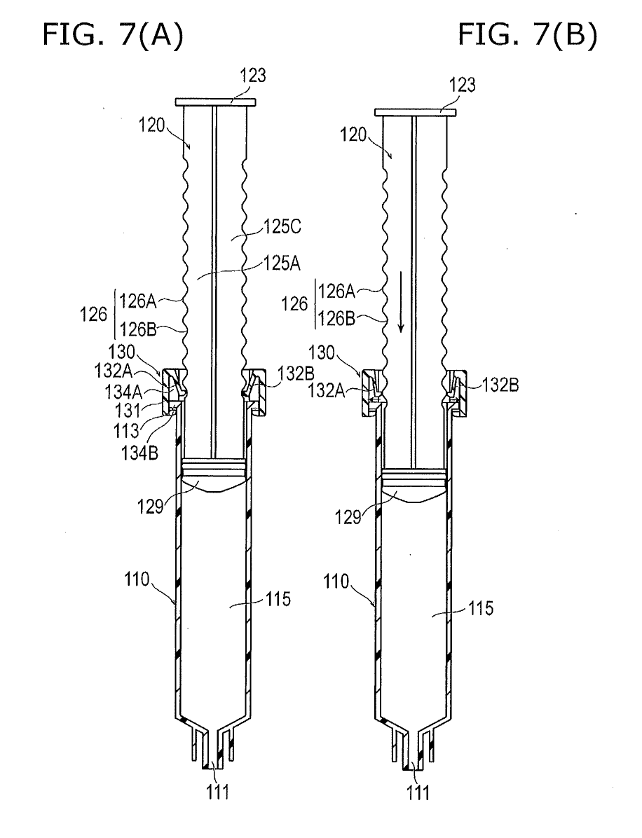

The gasket 129 is interlocked to the plunger forward end 122 so as to achieve a circumferentially rotatable fit, without being soldered to the latter. The gasket 129 is slidable in liquid-tight contact with an internal surface of the external cylinder 110. Therefore, by being translated integrally with the plunger main body 121 concurrently with advancing or retracting operation of the plunger main body 121, the gasket 129 plays the role of varying the volume of a fillable chamber 115 (see FIG. 7) inside the external cylinder 110 that is located on the forward side of the gasket 129 and is filled with a liquid.

The gasket 129 is not specifically restricted in regard of material. Preferable examples of the material include elastic materials, for instance, various rubber materials such as natural rubber, butyl rubber, isoprene rubber, butadiene rubber, styrene-butadiene rubber, silicone rubber, etc., various thermoplastic elastomers based on polyurethane, polyester, polyamide, olefin, styrene or the like, or mixtures thereof.

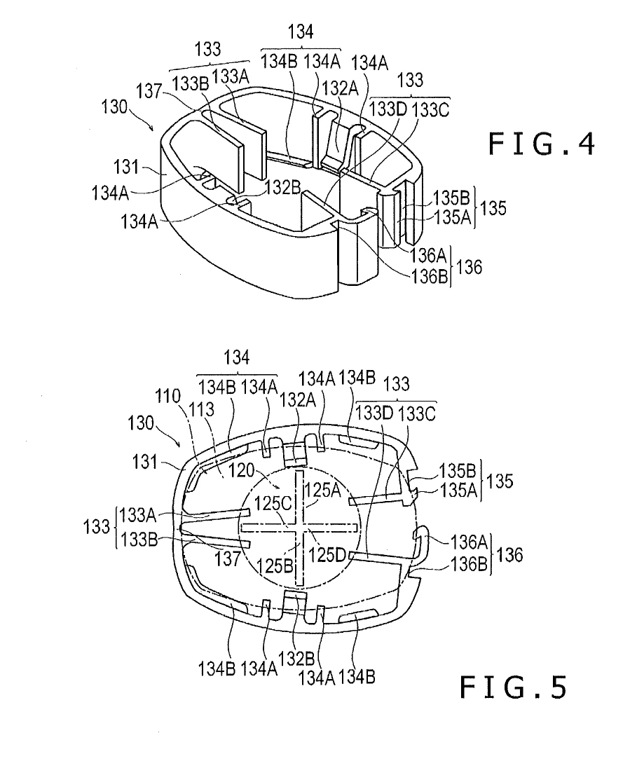

As shown in FIGS. 1, 4 and 5, the mounting fixture 130 includes: an annular member 131 in a discontinuous ring shape which is mountable to the external cylinder 110 so as to surround and clip the flange 113 of the external cylinder 110 from a direction crossing the longitudinal direction of the external cylinder 110; two protruding portions 132A and 132B contacting the concave and convex portion 126 of the plunger 120 inserted in the external cylinder 110; a rotation limiting member 133 limiting a rotation of the plunger 120 relative to the external cylinder 110; and flange-engaging members 134 engaging the flange 113. Further, the mounting fixture 130 is provided, at two discontinuous ends of the annular member 131, with a first interlock member 135 and a second interlock member 136 interlockable to each other, and a deformable portion 137 deformable so as to allow the first interlock member 135 and the second interlock member 136 to come closer to and away from each other.

The protruding portions 132A and 132B are formed on the inside of the annular member 131, make slidable contact with the concave and convex portion 126 of the plunger 120, and are retractable by deforming in an elastically bending manner so as to be spaced from the plunger 120. Therefore, each time the plunger 120 is advanced or retracted within the external cylinder 110, the protruding portions 132A and 132B are deformed to make contact with the plurality of convex portions 126A and concave portions 126B sequentially and in a slidable manner. It is to be noted that there is adopted a structure in which the protruding portions 132A and 132B themselves are not bent, or a structure in which the annular member 131 provided with the protruding portions 132A and 132B is deformable in a direction for spacing away from the plunger 120.

When the plunger 120 is translated forwards in a state in which the protruding portions 132A and 132B are each in contact with the concave portion 126B, each of the protruding portions 132A and 132B contacts the convex portion 126A while sliding on the concave and convex portion 126 and comes to contact the next concave portion 126B, whereby the liquid is discharged from the fillable chamber 115 through the flow port 111 to the exterior in a quantitative manner. In this instance, the product of the distance of a series of translations of each of the protruding portions 132A and 132B, from the concave portion 126B (contacted by it originally) through the convex portion 126A to the next concave portion 126B, by the inside sectional area of the external cylinder 110 becomes a target unit amount of liquid delivered. With this translating distance set to be an integer times the pitch of the graduations 114, the gasket 129 can be stopped on the graduations 114 on the basis of the unit amount of liquid delivered.

The rotation limiting member 133 is provided with four guide members 133A to 133D formed on the inside of the annular member 131. In a state in which the mounting fixture 130 is mounted to the external cylinder 110, the two guide members 133A and 133B are disposed in parallel to each other, with a predetermined spacing therebetween, so as to clip the blade member 125C of the plunger 120 in a direction perpendicular to the longitudinal direction of the external cylinder 110. Furthermore, in this state, the other two guide members 133C and 133D are disposed in parallel to each other, with a predetermined spacing therebetween, so as to clip the other blade member 125D in the direction perpendicular to the longitudinal direction of the external cylinder 110. Therefore, when the plunger 120 is advanced or retracted within the external cylinder 110, the guide members 133A to 133D function as walls which limit a rotation of the plunger 120 relative to the external cylinder 110 and hold the concave and convex portion 126 of the plunger 120 so that the concave and convex portion 126 would not be shifted in a rotating direction of the plunger 120 to come off the protruding portions 132A and 132B.

The flange-engaging members 134 are each provided with: a flange rear-side claw portion 134A contacting a rear-side surface of the flange 113 and protruding inward toward the plunger 120; and a flange forward-side claw portion 134B contacting a forward-side surface of the flange 113 and protruding from outside toward inside in conformity with the curved shape of the annular member 131. The flange 113 is clipped between the flange rear-side claw portions 134A and the flange forward-side claw portions 134B, whereby the mounting fixture 130 can be fixed to the flange 113. When the mounting fixture 130 is fixed to the flange 113 by the flange rear-side claw portions 134A and the flange forward-side claw portions 134B, it is ensured that the mounting fixture 130 can resist forces exerted thereon from the plunger 120 due to forward and rearward translations of the plunger 120, so that the mounting fixture 130 would not easily be disengaged from the external cylinder 110. It is to be noted that the numbers and shapes of the flange rear-side claw portions 134A and the flange forward-side claw portions 134B are not specifically restricted insofar as these members enable stable engagement of the mounting fixture 130 with the flange 113. In addition, the flange rear-side claw portions 134A may be configured to contact the blade members 125A and 125B of the plunger 120, thereby functioning as a rotation limiting member like the guide members 133A to 133D.

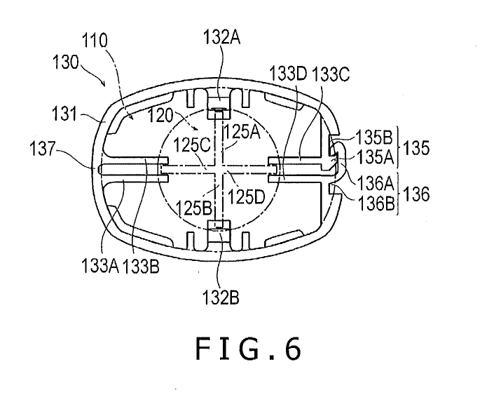

The deformable portion 137 is disposed at a position for substantially equally dividing the annular member 131 into a side on which the first interlock member 135 is provided and a side on which the second interlock member 136 is provided. In order that portions on opposite sides of the deformable portion 137 can easily come closer to and away from each other, the deformable portion 137 is formed thinner than the other portions of the annular member 131, to be lower than the other portions in flexural rigidity. Therefore, the annular member 131 is so configured that the first interlock member 135 and the second interlock member 136 can be opened a distance greater than the distance L (see FIG. 3 (B)) between adjacent tips of the blade members 125A to 125D of the plunger 120 at the time of mounting, whereby the plunger 120 and the external cylinder 110 can be accommodated inside the annular member 131 of the mounting fixture 130, and that the first interlock member 135 and the second interlock member 136 can be brought close to and interlocked to each other as shown in FIGS. 1 and 6. It is to be noted that while the deformable portion 137 is so formed as to provide a circular hole inside of the annular member 131 in this embodiment, the configuration of the deformable portion 137 is not specifically restricted. For instance, the deformable portion 137 may be formed so as to provide a circular hole outside of the annular member 131, or so as to provide a cut instead of a circular hole.

The first interlock member 135 and the second interlock member 136 are provided with a first claw portion 135A and a second claw portion 136A engaging each other, and a first hole portion 135B and a second hole portion 136B which allow easy bending of the first claw portion 135A and the second claw portion 136 for engagement with each other. Mutual engagement of the first claw portion 135A and the second claw portion 136A fixes the mounting fixture 130 to the flange 113 in such a manner as to surround the plunger 120.

The mounting fixture 130 is not particularly restricted in regard of material; for example, the aforementioned materials applicable to the external cylinder 110 can be used.

Now, operation of the syringe 100 according to the first embodiment will be described below.

First, before mounting of the mounting fixture 130, the external cylinder 110 and the plunger 120 in a state in which a liquid is accommodated in the external cylinder 110 and the plunger 120 is located on a rear side in the inside of the external cylinder 110, in other words, in a state in which the liquid can be discharged through the flow port 111 by translating the plunger 120 forwards, are prepared. It is to be noted that this state may be attained by a method in which the plunger 120 is pushed in to the deepest position in the inside of the external cylinder 110 and is then translated rearward, thereby sucking the liquid into the external cylinder 110 via the flow port 111. Alternatively, a prefilled-type external cylinder 110 having a liquid preliminarily accommodated in the external cylinder 110 may be used, together with a plunger 120.

Next, in order to mount the mounting fixture 130, portions near the first interlock member 135 and the second interlock member 136 are pulled and the annular member 131 is deformed with the deformable portion 137 as a fulcrum, so as to space the first interlock member 135 and the second interlock member 136 apart from each other, thereby resulting in a state in which the plunger 120 and the external cylinder 110 can be accommodated into the inside of the annular member 131 via an area between the first interlock member 135 and the second interlock member 136.

Subsequently, as shown in FIG. 5, the blade member 125C is located between the two guide members 133A and 133B spaced apart from each other by deformation of the deformable portion 137. It follows that the protruding portion 132A is opposed to the blade member 125A, the protruding portion 132B is opposed to the blade member 125B, and the blade member 125D is located between the two guide members 133C and 133D spaced apart from each other.

Next, the forces with which the portions of the annular member 131 are pulled are weakened, allowing the annular member 131 to return gradually into its original state. In this instance, such a positioning is conducted that the flange 113 is clipped between the flange rear-side claw portions 134A and the flange forward-side claw portions 134B. Then, the first claw portion 135A and the second claw portion 136A are mated and engaged with each other. It follows that, as shown in FIG. 6 and FIG. 7(A), the mounting fixture 130 is fixed to the external cylinder 110 in the state of wholly accommodating the plunger 120 so as to surround the plunger 120. In this state, the blade members 125C and 125D are clipped between the guide members 133A to 133D, and the protruding portions 132A and 132B abut on the concave portions 126B of the blade members 125A and 125B. It is to be noted that the mounting of the mounting fixture 130 may be conducted, not after suction of the liquid via the flow port 111 by the plunger 120 but before the suction.

Then, when the operating member 123 is pushed forward to translate the plunger 120 in order to deliver the liquid, the protruding portions 132A and 132B in contact with the concave portions 126B deform while sliding gradually on the concave and convex portion 126. Then, when the protruding portions 132A and 132B contact the convex portions 126A, as shown in FIG. 7(B), loads exerted on the protruding portions 132A and 132B are maximized, so that deformations of the protruding portions 132A and 132B are maximized. In this instance, a resisting force is exerted on the plunger 120, as well. As shown in FIG. 6, however, shifting of the plunger 120 in a rotating direction is restrained by the guide members 133A to 133D, and the two protruding portions 132A and 132B are so provided as to clip the plunger 120. Therefore, positional balancing is offered by resisting forces in opposite directions that are exerted from the two oppositely protruding portions 132A and 132B, whereby lateral shifting (shifting in a direction crossing the longitudinal direction) is also restrained. When the plunger 120 is advanced further, each of the protruding portions 132A and 132B advances to the next concave portion 126B, so that the loads on the protruding portions 132A and 132B are weakened, and the protruding portions 132A and 132B return into their original shapes. In this instance, a target unit amount of liquid is delivered through the flow port 111, and, at the operating member 123, a variation in a pressing force (pressure) is transmitted to the finger. Accordingly, the fact that the liquid has been delivered can be recognized through a sensation in the finger, without the need to visually check the graduations 114. Thereafter, the operating member 123 is further pushed, as required. This ensures that each of the protruding portions 132A and 132B slides while sequentially contacting the concave portion 126B and the convex portion 126A in this order. Accordingly, a target unit amount of liquid can be delivered through the flow port 111, while recognizing the concave portion 126B and the convex portion 126B through a sensation in the finger.

Thus, the syringe 100 according to the first embodiment includes: the external cylinder 110 fillable with a liquid therein; the plunger 120 translatable within the external cylinder 110 along the longitudinal direction of the external cylinder 110 and formed with the plurality of concave portions 126B and convex portions 126A aligned in the longitudinal direction; and the mounting fixture 130 in discontinuous ring shape that can be mounted so as to clip the external cylinder 110 from a direction crossing the longitudinal direction of the external cylinder 110. Therefore, the mounting fixture 130 can be mounted to the external cylinder 110 from a direction crossing the longitudinal direction of the external cylinder 110, by utilizing discontinuous portions of the mounting fixture 130. Accordingly, the mounting fixture 130 can be easily mounted in a short time and selectively only when necessary. In addition, the mounting fixture 130 is provided with the protruding portions 132A and 132B each making contact sequentially with the plurality of concave portions 126B and convex portions 126A in a slidable manner as the plunger 120 is translated, and the rotation limiting member 133 contacting the plunger 120 to limit a rotation of the plunger 120 relative to the external cylinder 110. Therefore, notwithstanding the mounting fixture 130 is of a selectively mounted type, rotation of the plunger 120 is limited by the rotation limiting member 133, whereby the contact of the protruding portions 132A and 132B with the concave portions 126B and the convex portions 126A is secured reliably, and, under this condition, the protruding portions 132A and 132B contact the concave portions 126B and the convex portions 126A of the plunger 120 sequentially. Accordingly, resisting forces exerted on the plunger 120 from the protruding portions 132A and 132B are transmitted to the finger, so that the fact that a target unit amount of liquid has been delivered can be recognized through a sensation in the finger.

In addition, the mounting fixture 130 is provided, at both discontinuous ends, with the first interlock member 135 and the second interlock member 136 interlockable to each other. Therefore, the mounting fixture 130 is fixed to the external cylinder 110 assuredly, and a stable liquid delivery operation can be achieved.

Furthermore, the annular member 131 has the deformable portion 137 which is lower in flexural rigidity than other portions of the annular member 131 and which is deformable so that the first interlock member 135 and the second interlock member 136 come closer to and away from each other. Therefore, the discontinuous portion of the mounting fixture 130 can be easily opened, in order to dispose the external cylinder 110 and the plunger 120 into the inside of the mounting fixture 130. This promises easier mounting and enhanced operability.

In addition, the rotation limiting member 133 has the pair of guide members 133A and 133B and the pair of guide members 133C and 133D configured so as to clip the plunger 120 in directions perpendicular to the longitudinal direction of the external cylinder 110. Therefore, rotation of the plunger 120 can be limited more assuredly, and a stabler liquid delivery operation can be achieved.

Furthermore, the pair of guide members 133A and 133B and the pair of guide members 133C and 133D can be spaced apart from each other through deformation of the mounting fixture 130 at the time of mounting the mounting fixture 130, and are located close to each other so as to clip the blade members 125C and 125D of the plunger 120 in the mounted state. Therefore, the plunger 120 can be easily disposed between the spaced-apart guide members 133A and 133B and the spaced-apart guide members 133C and 133D; in addition, after the mounting, rotation of the plunger 120 can be securely limited by the closely aligned guide members 133A and 133B and the closely aligned guide members 133C and 133D.

In addition, the mounting fixture 130 has the flange-engaging members 134 engaging the flange 113 at the edge of an opening portion for insertion of the plunger 120. Therefore, the mounting fixture 130 can resist the forces exerted thereon from the plunger 120 due to forward and rearward translations of the plunger 120. This ensures that the mounting fixture 130 would not easily be disengaged from the external cylinder 110, and that a stabler liquid delivery operation can be achieved.

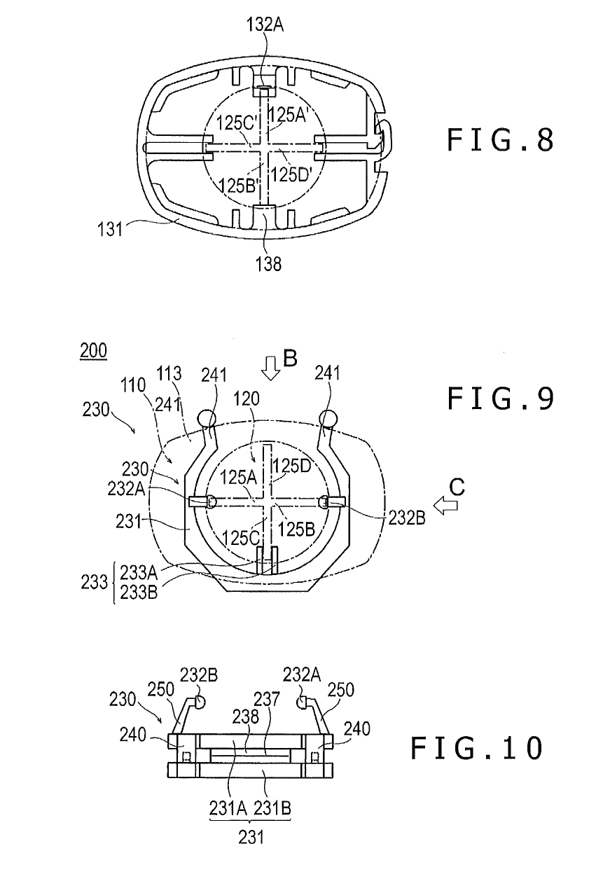

It is to be noted that combinations of the protruding portions 132A and 132B and the blade members 125A to 125D are not restricted to the aforementioned forms, and the combinations can be changed according to the use or the like. For instance, there may be adopted a configuration as shown in FIG. 8, in which only one protruding portion 132A is provided, all of four blade members 125A′ to 125D′ (concave and convex portion-forming members) are formed with concave and convex portions, and the blade members 125A′ to 125D′ have different pitches of the concave portions and the convex portions. In this case, the blade member to be brought into contact with the protruding portion 132A can be selected from the four blade members 125A′ to 125D′, whereby the unit amount by which the liquid can be delivered can be selected from four choices. Furthermore, in the case where only one protruding portion 132A is provided, a plate 138 extending from an annular member 131 to the tip of the blade member 125B′ may be formed at the inside of the annular member 131 opposite the protruding portion 132A, so as to fill up the gap between the blade member 125B′ and the annular member 131, thereby restraining lateral shifting of the plunger 120 due to a reaction force from the protruding portion 132A. The length of the plate 138 is preferably equal to or less than the distance from the inside of the annular member 131 to the tip of the blade member 125B′ in a stationary state.

In addition, the numbers of the flange forward-side claw portions 134B, the flange rear-side claw portions 134A and the guide members 133A to 133D can be changed, as required, according to the use or the like. For instance, the number of the guide members 133A to 133D is preferably at least two, in order that both rotation in one direction and rotation in the reverse direction can be limited. This number, however, is not specifically limited.

Furthermore, the guide members 133A to 133D may be provided with functions as flange rear-side claw portions, instead of separately providing the flange rear-side claw portions 134A.

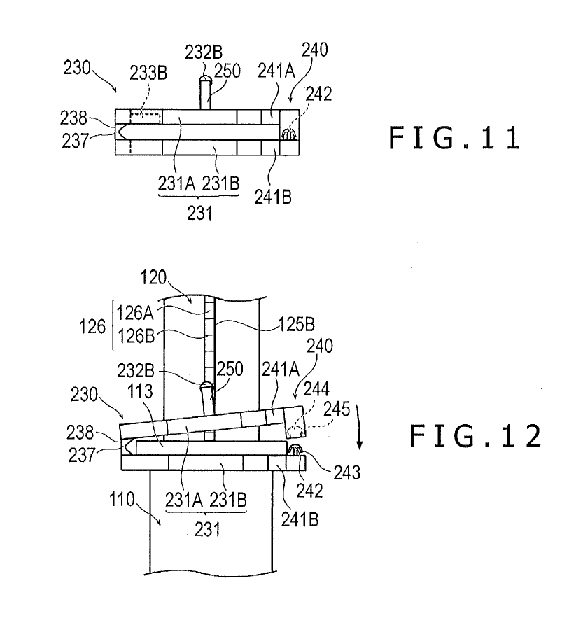

As shown in FIGS. 9 to 12, a syringe 200 according to a second embodiment includes: an external cylinder 110; a plunger 120 translatable within the external cylinder 110 along the longitudinal direction of the external cylinder 110; and a mounting fixture 230 mountable to the external cylinder 110. Incidentally, the external cylinder 110 and the plunger 120 are the same as those in the first embodiment above, and so they are denoted by the same reference signs as used above and descriptions of them are omitted.

The mounting fixture 230 is provided with: an annular member 231 in a discontinuous ring shape that is mountable to the external cylinder 110 so as to surround and clip a flange 113 of the external cylinder 110 from a direction crossing the longitudinal direction of the external cylinder 110; two protruding portions 232A and 232B contacting concave and convex portions 126 of the plunger 120 inserted in the external cylinder 110; and a rotation limiting member 233 limiting a rotation of the plunger 120 relative to the external cylinder 110.

The annular member 231 is provided with: a rear-side main body 231A contacting a rear-side surface of the flange 113 by being mounted to the external cylinder 110; and a forward-side main body 231B contacting a forward-side surface of the flange 113 and having an internal surface contacting an outer circumferential surface of the external cylinder 110. It is to be noted that each of the forward-side main body 231B and the rear-side main body 231A have two discontinuous ends thereof located separately, but, herein, such a shape is also defined as a ring shape.

The forward-side main body 231B and the rear-side main body 231A are interconnected by a connecting member 238 formed on a side roughly opposite the portion that is discontinuous in the circumferential direction of the ring shape. At the connecting member 238, there is formed a connecting member deformable portion 237 which is formed thinner than other portions of the connecting member 238 and which is lower than the other portions in flexural rigidity. Therefore, the forward-side main body 231B and the rear-side main body 231A can be opened toward the forward side and the rear side, with the connecting member 238 as a starting point.

Those surfaces of the forward-side main body 231B and the rear-side main body 231A which contact the external cylinder 110 may make whole-surface contact or may be machined to have concave and convex surfaces (or rugged surface) to make partial contact, for controlling the mounting force. In addition, the connecting member 238 may be in the form of a cut or may have a thin sheet structure projecting outward in a curved form.

The rear-side main body 231A and the forward-side main body 231B are formed at both their discontinuous ends with engaging member supports 241A and 241B which are opened away from each other and which extend so as to space away from the flange 113 after mounting.

As shown in FIG. 12, at an end portion of each engaging member support 241B of the forward-side main body 231B, there is formed a forward-side engaging projection 242 projecting toward the rear-side main body 231A, and the distal-side engaging projection 242 is formed with a projection-side claw portion 243. In addition, at an end portion of each engaging member support 241A of the rear-side main body 231A, there is formed a rear-side engaging recess 244 opening toward the forward-side main body 231B, and the rear-side engaging recess 244 is formed with a recess-side claw portion 245 protruding from an edge of the forward-side engaging recess 244. The forward-side engaging projection 242 and the rear-side engaging recess 244 constitute a main body engaging member 240 capable of mutual engagement. The forward-side engaging projection 242 can enter into the rear-side engaging recess 244, whereby the projection-side claw portion 243 is caught on the recess-side claw portion 245, and the forward-side engaging projection 242 engages the rear-side engaging recess 244. The size of the forward-side engaging projection 242 is equal to or less than the size of the rear-side engaging recess 244 so that the forward-side engaging projection 242 can be fitted into the rear-side engaging recess 244.

When the forward-side engaging projection 242 engages the rear-side engaging recess 244, the forward-side main body 231B and the rear-side main body 231A are fixed to the flange 113 so as to clip the flange 113 therebetween. In other words, the forward-side main body 231B and the rear-side main body 231A function as a flange-engaging member engaging the flange 113.

As shown in FIGS. 9 to 12, the protruding portions 232A and 232B are formed at tips of two protruding portion supports 250 extending obliquely from the rear-side main body 231A toward the inside (the plunger 120 side) and the rear side, and are each capable of contacting the concave and convex portion 126 of the plunger 120. With the protruding portions 232A and 232B contacting the concave and convex portions 126, the protruding portion supports 250 can retract through deforming while elastically deforming so that the protruding portions 232A and 232B are spaced apart from the plunger 120. Therefore, each time the plunger 120 is advanced or retracted within the external cylinder 110, the protruding portion supports 250 are deformed, and the protruding portions 232A and 232B make sliding contact sequentially with the plurality of convex portions 126A and concave portions 126B. It is to be noted that the protruding portion supports 250 may be pliable on a material basis or on a structural basis, and the protruding portions 232A and 232B themselves may be pliable on a material basis or on a structural basis.

The protruding portions 232A and 232B are in a semicircular shape projecting toward the inside (the plunger 120 side) in section along the longitudinal direction of the external cylinder 110, but other shapes may also be adopted. For example, the shape can be a triangular shape projecting toward the inside.

The rotation limiting member 233 is provided with two guide members 233A and 233B formed on the inside of the rear-side main body 231A. The two guide members 233A and 233B are disposed in parallel to each other, with a predetermined spacing therebetween, so as to clip the blade member of the plunger 120 from a direction perpendicular to the longitudinal direction of the external cylinder 110. Therefore, when the plunger 120 is advanced or retracted within the external cylinder 110, the guide members 233A and 233B function as walls that limit a rotation of the plunger 120 relative to the external cylinder 110 and that hold the concave and convex portions 126 of the plunger 120 so that the concave and convex portions 126 would not be shifted in a rotating direction of the plunger 120 to come off the protruding portions 232A and 232B. It is to be noted that the two guide members 233A and 233B are preferably formed in the vicinity of the connecting member 238 apart from the main body engaging member 240, in order that the blade member 125C of the plunger 120 can easily get between the two guide members 232A and 232B via the discontinuous portion. This specific configuration, however, is not required.

The mounting fixture 230 is not specifically restricted in regard of material; for instance, the aforementioned materials applicable to the external cylinder 110 can be used.

Now, operation of the syringe 200 according to the second embodiment will be described below.

First, before mounting of the mounting fixture 230, the external cylinder 110 and the plunger 120 in a state in which a liquid is accommodated in the external cylinder 110 and the plunger 120 is located on the rear side in the inside of the external cylinder 110, in other words, in a state in which the liquid can be discharged via the flow port 111 by translating the plunger 120 forwards, are prepared, like in the first embodiment.

Next, in order to mount the mounting fixture 230, in a state in which the forward-side engaging projection 242 and the rear-side engaging recess 244 are spaced from each other instead of engaging each other, as shown in FIG. 12, the positions of the guide members 233A and 233B are aligned in relation to the blade member 125C, whereas the positions of the protruding portions 232A and 232B are aligned in relation to the blades 125A and 125B, and, concurrently with these operations, the rear-side main body 231A and the forward-side main body 231B are fitted into the external cylinder 110 so as to clip the flange 113, from the side on which the main body engaging member 240 is provided. In this instance, since the engaging portion supports 241A and 241B are opening to the outside, the external cylinder 110 can be easily guided into the inside of the mounting fixture 230. In addition, since the forward-side main body 231B and the rear-side main body 231A are movable toward the forward side and the rear side with the connecting member 238 as a fulcrum, the forward-side main body 231B and the rear-side main body 231A can be inserted more easily while moving them appropriately.

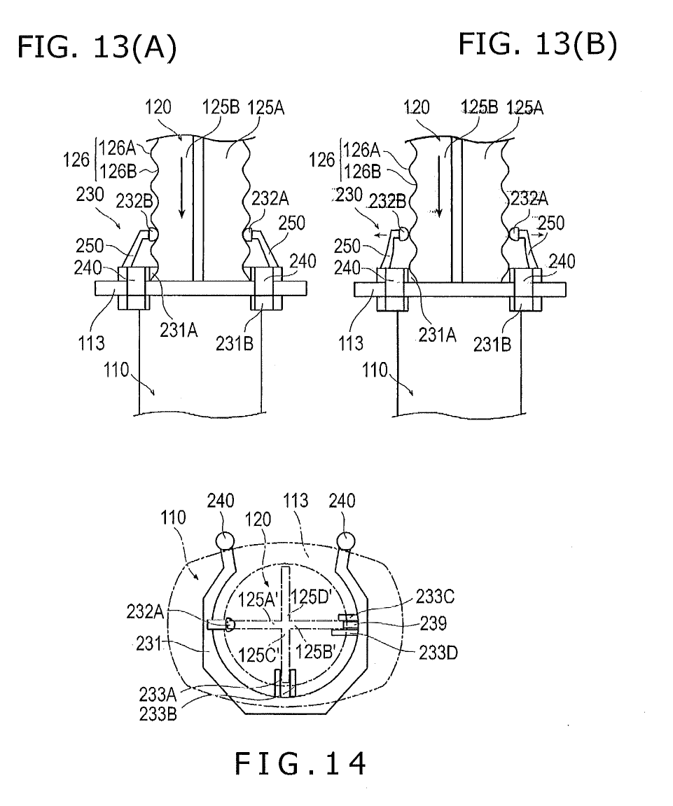

Subsequently, the forward-side engaging projection 242 is pushed rearward, whereas the rear-side engaging recess 244 is pushed forward, whereby the forward-side engaging projection 242 and the rear-side engaging recess 244 are engaged with each other. The projection-side claw portion 243 is inserted into the rear-side engaging recess 244 while being deformed by being blocked by an entrance of the rear-side engaging recess 244. After the insertion, the projection-side claw portion 243 returns into its original shape. After this, even if a forward force is exerted on the forward-side engaging projection 242, the projection-side claw portion 243 is caught on the recess-side claw portion 245, so that disengagement would not easily occur. As a result, the mounting fixture 230 is fixed to the external cylinder 110 in the state of accommodating the plunger 120 so as to surround the plunger 120, as shown in FIG. 13(A). When this state is established, the blade member 125C is clipped between the guide members 233A and 233B, and the protruding portions 232A and 232B abut on the concave portions 126B of the blade members 125A and 125B. It is to be noted that the mounting of the mounting fixture 230 may be performed, not after suction of the liquid through the flow port 111 by the plunger 120 but before the suction.

Then, when the operating member 123 is pushed forward to translate the plunger 120 in order to deliver the liquid, the protruding portions 232A and 232B contacting the concave portions 126B gradually slide on the concave and convex portions 126, whereby the protruding portion supports 250 are deformed through bending. Then, when the protruding portions 232A and 232B contact the convex portions 126A, as shown in FIG. 13(B), loads exerted on the protruding portions 232A and 232B are maximized, so that deformations of the protruding portion supports 250 are maximized. In this instance, resisting forces act on the plunger 120, as well. Since shifting of the plunger 120 in a rotating direction is restrained by the guide members 233A and 233B and the two protruding portions 232A and 232B are so provided as to clip the plunger 120, however, positional balancing is offered by resisting forces in opposite directions that are exerted from the two protruding portions 232A and 232B opposite each other, so that lateral shifting (shifting in a direction crossing the longitudinal direction) is restrained, as well. When the plunger 120 is advanced further, the protruding portions 232A and 232B each advance to the next concave portion 126B, whereby the loads on the protruding portions 232A and 232B are weakened, so that the protruding portions 232A and 232B return into their original positions. In this instance, a target unit amount of liquid is delivered through the flow port 111; in addition, at the operating member 123, a variation in the pressing force (pressure) is transmitted to the finger. Therefore, the fact that the liquid has been delivered can be recognized through a sensation in the finger, without the need to visually check the graduations 114. Thereafter, the operating member 123 is pushed further, as required, whereby the protruding portions 232A and 232B each slide while sequentially contacting the concave portion 126B and the convex portion 126A in this order. Accordingly, a target unit amount of liquid can be delivered through the flow port 111, while recognizing the concave portion 126B and the convex portion 126A through a sensation in the finger.

Thus, according to the syringe 200 in the second embodiment, the two discontinuous ends of the mounting fixture 230 are located apart from each other in the state in which the mounting fixture 230 is mounted to the external cylinder 110, which promises easy insertion of the external cylinder 110 and the plunger 120 into the inside of the mounting fixture 230 and enhanced operability. In addition, both discontinuous ends are located apart from each other and need not be interlocked to each other. Therefore, even when an external cylinder 110 or plunger 120 different in size (particularly, outside diameter) is inserted, the discontinuous portion can deform so as to open wide according to the shape of the external cylinder 110 or plunger 120. Thus, a single mounting fixture 230 can cope with a variety of external cylinders 110 and plungers 120.

Furthermore, the mounting fixture 230 is provided with: the rear-side main body 231A disposed on the rear side of the flange 113; the forward-side main body 231B disposed on the forward side of the flange 113; the connecting member 238 interconnecting the forward-side main body 231B and the rear-side main body 231A; and the main body engaging member 240 capable of interlocking the forward-side main body 231B and the rear-side main body 231A. Therefore, with the flange 113 clipped by the forward-side main body 231B and the rear-side main body 231A and with the forward-side main body 231B and the rear-side main body 231A interlocked to each other by the main body engaging member 240, the mounting fixture 230 is fixed to the external cylinder 110 assuredly, and a stable liquid delivery operation can be achieved.

In addition, the connecting member 238 is provided with the connecting member deformable portion 237 which is lower than other portions of the connecting member 238 in flexural rigidity and which is deformable so that the forward-side main body 231B and the rear-side main body 231A can come closer to and away from each other. Therefore, the forward-side main body 231B and the rear-side main body 231A can be easily opened so as to space apart from each other, which promises easy mounting of the mounting fixture 230 and enhanced operability.

It is to be noted that combinations of the protruding portions 232A and 232B and the blade members 125A to 125D are not restricted to the aforementioned forms, and the combinations can be changed according to the use or the like. For example, a configuration as shown in FIG. 14 can be adopted, in which only one protruding portion 232A is provided, all of four blade members 125A′ to 125D′ (concave and convex portion-forming members) are formed with concave and convex portions, and the blade members 125A′ to 125D′ have different pitches of concave portions and convex portions. In this case, the blade member to be brought into contact with the protruding portion 232A can be selected from the four blade members 125A′ to 125D′, so that the unit amount by which the liquid is delivered can be selected from four choices. In addition, in the case where only one protruding portion 232A is provided, a configuration may be adopted in which a plate 239 extending from the annular member 231 to the tip of the blade member 125B′ on the inside of the annular member 231 opposite the protruding portion 232A is formed so as to fill up the gap between the blade member 125B′ and the annular member 231, whereby lateral shifting of the plunger 120 by a reaction force from the protruding portion 231A is prevented. The length of the plate 239 is preferably equal to or less than the distance from the inside of the annular member 231 to the tip of the blade member 125B′ in a stationary state.

In addition, the number and shapes of the guide members can be changed, as required, according to the use or the like. For instance, as shown in FIG. 14, guide members 233C and 233D may be provided. In this case, the guide member 233C on one side near the side from which the plunger 120 is inserted may be formed to be shorter, for facilitating the insertion of the blade member 125B′.

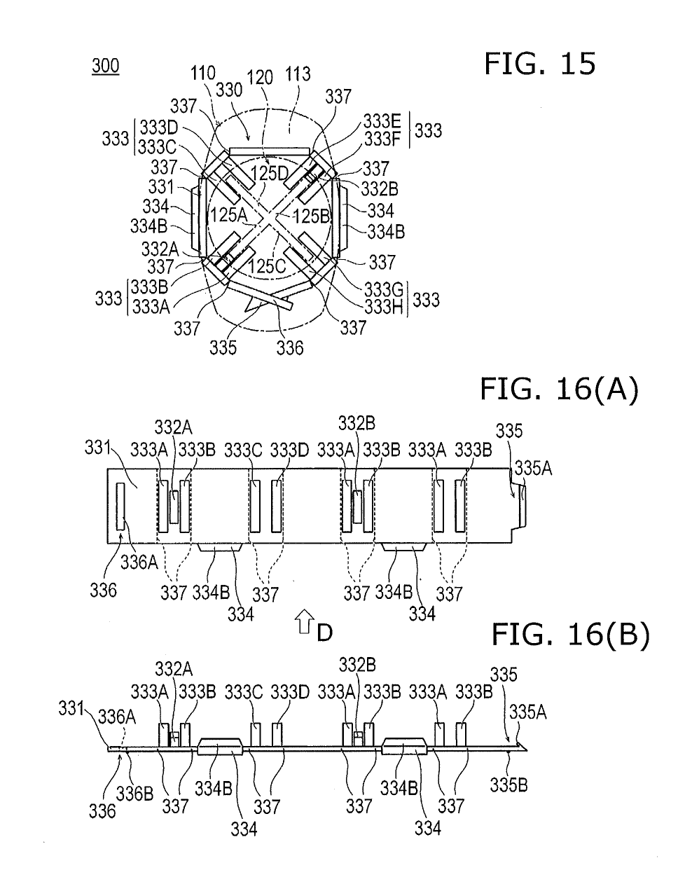

As shown in FIG. 15, a syringe 300 according to a third embodiment includes: an external cylinder 110; a plunger 120 translatable within the external cylinder 110 along the longitudinal direction of the external cylinder 110; and a mounting fixture 330 mountable to the external cylinder 110. It is to be noted that since the external cylinder 110 and the plunger 120 are the same as in the first embodiment above, they are denoted by the same reference signs as used above and descriptions of them are omitted.

As shown in FIGS. 15 to 17, the mounting fixture 330 is provided with: a flat-shaped member 331 (annular member) mountable to the external cylinder 110 so as to surround and clip a flange 113 of the external cylinder 110 from a direction crossing the longitudinal direction of the external cylinder 110; two protruding portions 332A and 332B contacting concave and convex portions 126 of the plunger 120 inserted in the external cylinder 110; a rotation limiting member 333 limiting a rotation of the plunger 120 relative to the external cylinder 110; and flange-engaging members 334 engaging the flange 113. Further, the mounting fixture 330 is provided with a first interlock member 335 and a second interlock member 336 which are formed at both side ends located on opposite sides at the flat-shaped member 331 and which are interlockable to each other. In addition, a plurality of folds 337 (deformable portions) deformable by bending are formed between the first interlock member 335 and the second interlock member 336 so that the first interlock member 335 and the second interlock member 336 can come close to and away from each other.

The flat-shaped member 331 has a roughly rectangular resin-made flat plate provided with the folds 337, which are lower in flexural rigidity than other portions of the flat-shaped member 331. The folds 337 are provided in eight and in an aligned manner, whereby the flat-shaped member 331 is divided into nine parts. When bent at the folds 337, the flat-shaped member 331 is deformed so that the parts are aligned in a ring shape, allowing the plunger 120 accommodated therein. While the folds 337 are each formed of a cut provided in an outer circumferential surface of the flat-shaped member 331, on a structural basis, the structure of the folds 337 is not specifically restricted insofar as the folds 337 allows bending. For instance, the folds 337 may have a groove structure where thickness of the flat-shaped member 331 is smaller than at other portions of the flat-shaped member 331; or, alternatively, the folds 337 may each be a perforation including some through-holes at intermediate portions thereof. In addition, the number of the folds 337 is not limited to eight. For example, the number of the folds 337 may be four so that the flat-shaped member 331 may be deformed into a roughly quadrilateral ring shape.

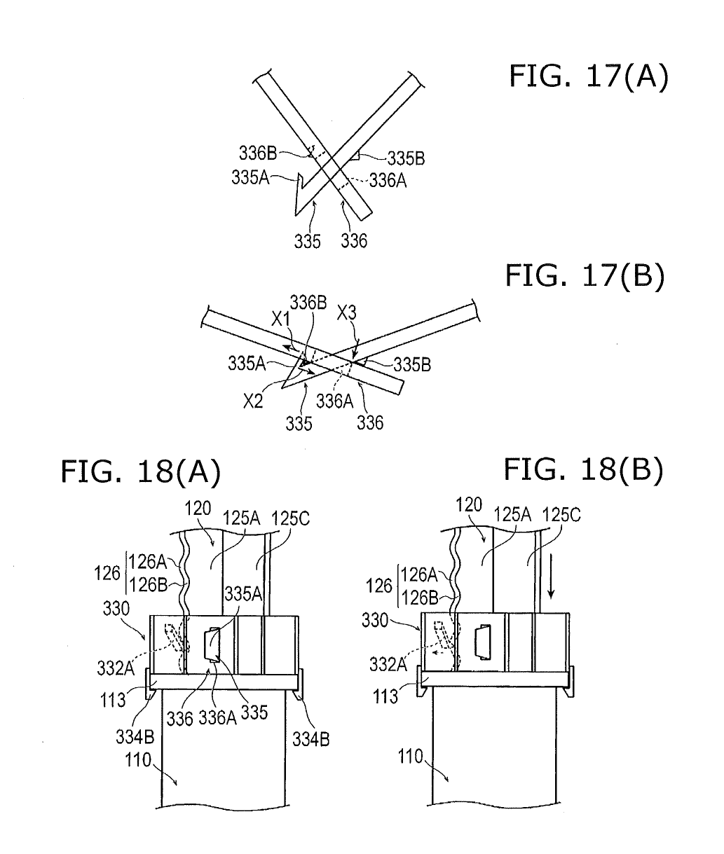

As shown in FIG. 17(A), the first interlock member 335 is provided with: a hook-shaped end claw portion 335A formed to protrude to the inner side at an end portion; and an end stopper 335B formed to protrude to the outer side. The second interlock member 336 is provided with: an end claw hole 336A to be penetrated by the end claw portion 335A; and an engaging stopper 336 protruding to the outer side for engagement with the end claw portion 335A. With the first interlock member 335 and the second interlock member 336 interlocked to each other, the flat-shaped member 331 is so fixed as to surround the plunger 120 along the circumferential direction on the rear side of the flange 113. The width of the tip of the end claw portion 335A is sufficiently smaller than the width of the end claw hole 336A. In this case, when the end claw portion 335A is fitted to and engaged with the engaging stopper 336A, as shown in FIG. 17(B), the end claw portion 335A is inserted gaplessly and obliquely in relation to the end claw hole 336A, and makes contact with an edge of the end claw hole 336A, whereby chattering is restrained. This ensures that the end claw portion 335A is restrained from shifting in any of directions X1 and X2 (see arrows in FIG. 7(B)). In addition, the end stopper 335B restrains the end claw portion 335A from being disengaged from the engaging stopper 336B due to an excessive insertion of the end claw portion 335A in a claw tip direction X3 (see arrow in FIG. 17(B)). Consequently, the mounting fixture 330 can be restrained from coming out of position (dropping) due to an artificial load exerted on the mounting fixture 330 after mounting thereof.

As shown in FIGS. 15 and 16, the protruding portions 332A and 332B are formed on the inside of the flat-shaped member 331, are capable of contacting concave and convex portions 126 of the plunger 120, and are retractable through deformation in the manner of elastically bending so as to come away from the plunger 120. Therefore, each time the plunger 120 is advanced or retracted within the external cylinder 110, the protruding portions 332A and 332B are deformed, to make sliding contact sequentially with a plurality of convex portions 126A and concave portions 126B. It is to be noted that a structure may be adopted in which the protruding portions 332A and 332B themselves are not pliable, or a structure may be adopted in which the flat-shaped member 331 provided with the protruding portions 332A and 332B is deformable in a direction away from the plunger 120.

The rotation limiting member 333 is provided with eight guide members 333A to 333H formed on the inside of the flat-shaped member 331. In a state in which the mounting fixture 330 is mounted to the external cylinder 110, each of pairs of the guide members 333A to 333H are disposed in parallel, with a predetermined spacing therebetween, so as to clip a corresponding one of the four blade members 125A to 125D from a direction perpendicular to the longitudinal direction of the external cylinder 110. Therefore, when the plunger 120 is advanced or retracted within the external cylinder 110, the guide members 333A to 333H function as walls which limit a rotation of the plunger 120 relative to the external cylinder 110 and which hold the concave and convex portions 126 of the plunger 120 so that the concave and convex portions 126 would not be disengaged from the protruding portions 332A and 332B through shifting in a rotating direction of the plunger 120. The number and shapes of the guide members 333A to 333H are not specifically restricted, insofar as they make it possible to prevent the shifting in a rotating direction.

The flange-engaging member 334 has flange forward-side claw portions 334B contacting a forward-side surface of the flange 113 and protruding from outside toward inside in conformity with a curved shape of the flat-shaped member 331. The flange 113 is clipped between the flange forward-side claw portions 334B and the guide members 333A to 333H, which contact a rear-side surface of the flange 113, whereby the mounting fixture 330 can be fixed to the flange 113. When the mounting fixture 330 is fixed to the flange 113 by the guide members 333A to 333H and the flange forward-side claw portions 334B, the mounting fixture 330 can resist forces exerted from the plunger 120 due to forward and rearward translations of the plunger 120, so that the mounting fixture 330 would not easily be disengaged from the external cylinder 110. It is to be noted that the number and shapes of the flange forward-side claw portions 334B are not specifically restricted, insofar as the flange forward-side claw portions 334 can stably engage the flange 113. In addition, each of the guide members 333A to 333H has a function of fixing the mounting fixture 330 by contacting the rear-side surface of the flange 113. In this case, all the guide members 333A to 333H may not necessarily contact the flange 113, and the guide members 333A to 333H may be being separate from the flange 113, insofar as the mounting fixture 330 can be fixed. Furthermore, since a forward-side end face of the flat-shaped member 331 contacts a rear-side surface of the flange 113, the flange forward-side claw portion 334B and the flat-shaped member 331 may constitute a flange-engaging member 334.

The mounting fixture 330 is not specifically restricted in regard of material; for example, the aforementioned materials applicable to the external cylinder 110 can be used.

Now, operation of the syringe 300 according to the third embodiment will be described below.

First, before mounting of the mounting fixture 330, the external cylinder 110 and the plunger 120 in a state in which a liquid is accommodated in the external cylinder 110 and the plunger 120 is located on the rear side in the inside of the external cylinder 110, in other words, in a state in which the liquid can be discharged through a flow port 111 by translating the plunger 120 forward, are prepared, like in the first embodiment.

Next, in order to mount the mounting fixture 330, the position of the plunger 120 in a rotating direction is determined, then, while bending the flat-shaped member 331 in a flat plate form by utilizing the folds 337, the protruding portions 332A and 332B are abutted on the concave and convex portions 126 of the blade members 125A and 125B, and the flange 113 is clipped by the flange forward-side claw portions 334B and the guide members 333A to 333H. The plunger 120 is surrounded with the flat-shaped member 331, and those parts of the flat-shaped member 331 at which the flange forward-side claw portions 334B are provided are pressed against the plunger 120. As a result, a surface formed with the end claw hole 336A and a surface formed with the end claw portion 335A intersect at a wide angle (an angle proximate to a right angle), as shown in FIG. 17(A), and the end claw portion 335A can be made to penetrate the end claw hole 336A. Thereafter, when the pressing of the flat-shaped member 331 is weakened and the surface formed with the end claw portion 335A and the surface formed with the end claw hole 336A come to intersect at a narrow angle, as shown in FIG. 17(B), the tip of the end claw portion 335A is caught on the engaging stopper 336B, whereby the first interlock member 335 and the second interlock member 336 are interlocked, and the mounting fixture 330 is completely fixed to the external cylinder 110. When this state is established, as shown in FIGS. 15 and 18(A), the blade members 125A to 125D are clipped between the guide members 333A to 333H, and the protruding portions 332A and 332B abut on the concave portions 126B of the blade members 125A and 125B. In this instance, the end claw portion 335A is in gapless contact in relation to the internal space of the end claw hole 336A, so that the end claw portion 335A would not easily be disengaged from the end claw hole 336A. Moreover, as shown in FIG. 17(B), the end stopper 335B restrains the end claw portion 335A from coming off the engaging stopper 336B due to an excessive insertion of the end claw portion 335A in a claw tip direction, whereby the mounting fixture 330 can be restrained from coming out of position (dropping) due to an artificial load exerted on the mounting fixture 330 after the mounting thereof. It is to be noted that the mounting of the mounting fixture 330 may be performed, not after suction of a liquid through the flow port 111 by the plunger 120 but before the suction.

Then, when the operating member 123 is pushed forward to translate the plunger 120 in order to deliver the liquid, the protruding portions 332A and 332B making contact with the concave portions 126B gradually slide on the concave and convex portions 126, while deforming. Then, when the protruding portions 332A and 332B contact the convex portions 126A, as shown in FIG. 18(B), loads exerted on the protruding portions 332A and 332B are maximized, so that deformations of the protruding portions 332A and 332B are maximized. In this instance, resisting forces act on the plunger 120, as well. Since shifting of the plunger 120 in a rotating direction is restrained by the guide members 333A to 333H and the two protruding portions 332A and 332B are so provided as to clip the plunger 120, however, positional balancing is offered by resisting forces in opposite directions that are exerted from the two protruding portions 332A and 332B opposite each other, so that lateral shifting (shifting in a direction crossing the longitudinal direction) is restrained, as well. When the plunger 120 is advanced further, the protruding portions 332A and 332B each advance to the next concave portion 126B, whereby the loads on the protruding portions 332A and 332B are weakened, so that the protruding portions 332A and 332B return into their original shapes. In this instance, a target unit amount of liquid is delivered through the flow port 111; in addition, at the operating member 123, a variation in the pressing force (pressure) is transmitted to the finger. Therefore, the fact that the liquid has been delivered can be recognized through a sensation in the finger, without the need to visually check graduations 114. Thereafter, the operating member 123 is pushed further, as required, whereby the protruding portions 332A and 332B each slide while sequentially contacting the concave portion 126B and the convex portion 126A in this order. Accordingly, a target unit amount of liquid can be delivered through the flow port 111, while recognizing the concave portion 126B and the convex portion 126A through a sensation in the finger.

Thus, the syringe 300 according to the third embodiment includes: the external cylinder 110 fillable with a liquid therein; the plunger 120 translatable within the external cylinder 110 along the longitudinal direction of the external cylinder 110 and formed with the plurality of concave portions 126B and convex portions 126A aligned in the longitudinal direction; and the mounting fixture 330 provided with the flat-shaped member 331 in a flat plate form that is mountable to the external cylinder 110 by being deformed into a ring shape. Therefore, the mounting fixture 330 can be mounted to the external cylinder 110 from a direction crossing the longitudinal direction of the external cylinder 110, by utilizing a discontinuous part of the mounting fixture 330. Accordingly, the mounting fixture 330 can be easily mounted in a short time, and can be mounted selectively only when necessary.

In addition, the mounting fixture 330 is provided with the protruding portions 332A and 332B each making contact sequentially with the plurality of concave portions 126B and convex portions 126A in a slidable manner as the plunger 120 is translated, and the rotating limiting member 333 contacting the plunger 120 to limit a rotation of the plunger 120 relative to the external cylinder 110. Therefore, notwithstanding the mounting fixture 330 is of a selectively mounted type, rotation of the plunger 120 is limited by the rotation limiting member 333, whereby the contact of the protruding portions 332A and 332B with the concave portions 126B and the convex portions 126A is secured reliably, and, under this condition, the protruding portions 332A and 332B contact the concave portions 126B and the convex portions 126A of the plunger 120 sequentially. Accordingly, resisting forces exerted on the plunger 120 from the protruding portions 332A and 332B are transmitted to the finger, so that the fact that a target unit amount of liquid has been delivered can be recognized through a sensation in the finger. In addition, the mounting fixture 330 can be kept housed in a compact flat plate form until put to use, so that packaging and storage thereof is easy, and a reduction in production cost can also be achieved.

In addition, the mounting fixture 330 is provided, at both ends of the flat-shaped member 331, with the first interlock member 335 and the second interlock member 336 interlockable to each other. Therefore, the mounting fixture 330 is fixed to the external cylinder 110 assuredly, and a stable liquid delivery operation can be achieved.

Furthermore, the mounting fixture 330 has the flange-engaging members 334 for engagement with the flange 113 at the edge of the opening portion of the external cylinder 110 in which the plunger 120 is inserted. Therefore, the mounting fixture 330 can resist forces exerted thereon from the plunger 120 due to forward and rearward translations of the plunger 120. Accordingly, the mounting fixture 330 would not easily be disengaged from the external cylinder 110, and a stable liquid delivery operation can be achieved.

In addition, the flat-shaped member 331 is provided with the folds 337 (deformable portion) which are lower in flexural rigidity than other portions of the flat-shaped member 331. Therefore, the flat-shaped member 331 can be easily deformed into a ring shape, which promises easy mounting and enhanced operability.

It is to be noted that combinations of the protruding portions 332A and 332B and the blade members 124A to 125D are not restricted to the aforementioned forms, and the combinations can be changed according to the use or the like. For instance, as has been described in the first embodiment and the second embodiment, there may be adopted a configuration in which only one protruding portion is provided, all of four blade members are formed with concave and convex portions, and the blade members have different pitches of the concave portions and convex portions. In this case, the blade member to be brought into contact with the protruding portion can be selected from the four blade members, whereby the unit amount by which the liquid can be delivered can be selected from four choices.

Furthermore, the numbers of the flange forward-side claw portions 334B, the guide members 333A to 333H and the folds 337 can be changed, as required, according to the use or the like. In addition, a configuration may be adopted in which the flat-shaped member 331 is not provided with the folds 337 (deformable portions), and the flat-shaped member 331 is deformed into a ring shape which curves as a whole.

Furthermore, the first interlock member 335 and the second interlock member 336 may be configured differently from the aforementioned, insofar as they are interlockable to each other.

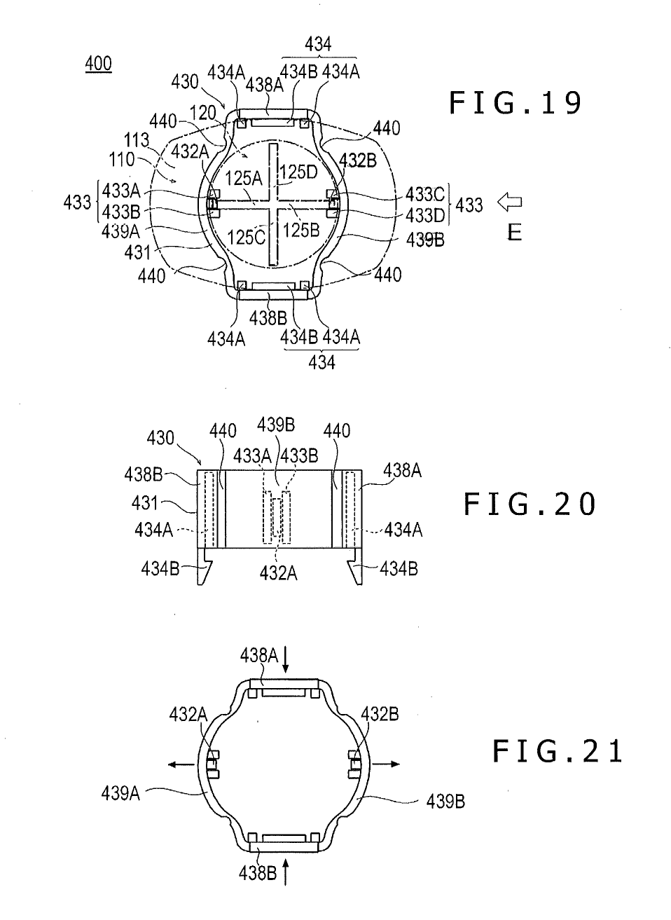

As shown in FIGS. 19 and 20, a syringe 400 according to a fourth embodiment includes: an external cylinder 110; a plunger 120 translatable within the external cylinder 110 along the longitudinal direction of the external cylinder 110; and a mounting fixture 430 mountable to the external cylinder 110. It is to be noted that since the external cylinder 110 and the plunger 120 are the same as in the first embodiment above, they are denoted by the same reference signs as used above and descriptions of them are omitted.

The mounting fixture 430 is provided with: an annular member 431 in a ring shape that is mountable to the external cylinder 110 so as to surround a flange 113 of the external cylinder 110; two protruding portions 432A and 432B contacting concave and convex portions 126 of the plunger 120 inserted in the external cylinder 110; a rotation limiting member 433 limiting a rotation of the plunger 120 relative to the external cylinder 110; and flange-engaging members 434 engaging the flange 113.

The annular member 431 is provided with: translating members 439A and 439B which are provided opposite to each other and are accompanied by protruding portions 432A and 432B disposed on the inside thereof; pressing members 438A and 438B provided in pair for pressing by fingers; and bending members 440 (deformable portions) each provided between the translating member 439A and 439B and the pressing member 438A and 438B and formed to be curved toward the outside of the ring shape.

When the mounting fixture 430 is mounted, the pressing members 438A and 438B are located to be more spaced outward from the plunger 120 than the translating members 439A and 439B. The pressing members 438A and 438B themselves are formed with such a strength as not to be easily bent even if pressed by a finger. Bending members 440 are formed to be thinner, and lower in flexural rigidity, than other portions of the annular member 431. This structure ensures that when forces are externally exerted so as to bring the pair of pressing members 438A and 438B toward each other, the forces transmitted act to deform the translating members 439A and 439B toward the outside of the ring shape through bending. It is to be noted that while the pressing members 438A and 438B are formed from the same resin material as the other portions of the mounting fixture 430, the pressing members 438A and 438B may be formed from a different material. Furthermore, the pressing members 438A and 438B may be formed at outside surfaces thereof with a rugged structure for preventing slipping of fingers thereon. The rugged structures can be provided, for example, by attaching other members to the pressing members 438A and 438B. The bending members 440 may have a structure in which the material thickness is partially reduced by forming a cut, without being curved.

The protruding portions 432A and 432B are formed on the inside of the translating members 439A and 439B, are capable of contacting concave and convex portions 126 of the plunger 120, and are retractable through deformation in the manner of elastically bending so as to come away from the plunger 120. Therefore, each time the plunger 120 is advanced or retracted within the external cylinder 110, the protruding portions 432A and 432B are deformed, to make contact sequentially with a plurality of convex portions 126A and concave portions 126B in a slidable manner. It is to be noted that there may be adopted a structure in which the protruding portions 432A and 432B themselves are not pliable, or a structure in which the annular member 431 provided with the protruding portions 432A and 432B is deformable in a direction away from the plunger 120.

The rotation limiting member 433 has four guide members 433A to 433D formed on the inside of the translating members 439A and 439B. In a state in which the mounting fixture 430 is mounted to the external cylinder 110, the two guide members 433A and 433B are disposed in parallel, with a predetermined spacing therebetween, so as to clip a blade member 125A of the plunger 120 from a direction perpendicular to the longitudinal direction of the external cylinder 110. The other two guide members 433C and 433D are disposed in parallel, with a predetermined spacing therebetween, so as to clip another blade member 125B from a direction perpendicular to the longitudinal direction of the external cylinder 110. Therefore, when the plunger 120 is advanced or retracted within the external cylinder 110, the guide members 433A to 433D function as walls which limit a rotation of the plunger 120 relative to the external cylinder 110 and which hold the concave and convex portions 126 of the plunger 120 so as to prevent the concave and convex portions 126 from being disengaged from the protruding portions 432A and 432B through shifting in a rotating direction of the plunger 120.

The flange-engaging member 434 is provided with: a flange rear-side claw portion 434A contacting a rear-side surface of the flange 113 and protruding toward the plunger 120; and a flange forward-side claw portion 434B contacting a forward-side surface of the flange 113 and protruding from outside toward inside in conformity with a curved shape of the annular member 431. With the flange 113 clipped between the flange rear-side claw portions 434A and the flange forward-side claw portions 434B, the mounting fixture 430 can be fixed to the flange 113. When the mounting fixture 430 is fixed to the flange 113 by the flange rear-side claw portions 434A and the flange forward-side claw portions 434B, the mounting fixture 430 can resist forces exerted thereon from the plunger 120 due to forward and rearward translations of the plunger 120, so that the mounting fixture 430 would not easily be disengaged from the external cylinder 110.

In addition, the flange forward-side claw portion 434B is formed in an oblique shape such that the protruding amount of protrusion decreases along a direction toward the tip (see FIG. 20), and has such a strength and structure that it can be warped, with the point at which it joins to the annular member 431 as a fulcrum. Therefore, when the flange forward-side claw portions 434B are moved from the rear side toward the forward side of the flange 113, the flange forward-side claw portions 434B are each warped, with the point at which it joins to the annular member 431 as a fulcrum, whereby the flange 113 can be easily inserted between the flange rear-side claw portions 434A and the flange forward-side claw portions 434B. It is to be noted that the numbers and shapes of the flange rear-side claw portions 434A and the flange forward-side claw portions 434B are not specifically restricted, insofar as these portions can stably engage the flange 113. In addition, a configuration may be adopted in which the flange rear-side claw portions 434A are not provided, and the flange 113 is fixed by clipping it between a forward-side surface of the annular member 431 and the flange forward-side claw portions 434B.

Now, operation of the syringe 400 according to the fourth embodiment will be described below.

First, before mounting of the mounting fixture 430, the external cylinder 110 and the plunger 120 in a state in which a liquid is accommodated in the external cylinder 110 and the plunger 120 is located on the rear side in the inside of the external cylinder 110, in other words, in a state where the liquid can be discharged through a flow port 111 by translating the plunger 120 forward, are prepared, like in the first embodiment.

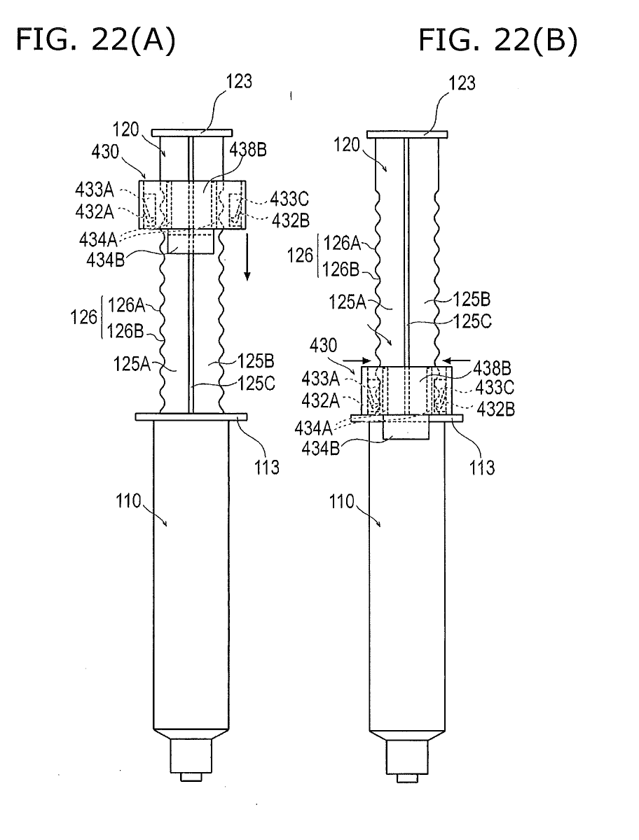

Next, in order to mount the mounting fixture 430, the pressing members 438A and 438B are nipped by one hand, and are pressed toward the inside from both sides, as shown in FIG. 21. In this instance, the translating members 439A and 439B are deformed outward in an arcuate form as a whole, and the protruding portions 432A and 432B are also moved outward. Then, while maintaining this state, the mounting fixture 430 is fitted over the syringe 400 held by the other hand, in the manner of mounting the mounting fixture 430 on an operating member 123 of the syringe 400 from the rear side, as shown in FIG. 22(A). The mounting fixture 430 is moved to just the rear side of the flange 113, the positions of the concave and convex portions 126 of the blade members are adjusted to the guide members 434A to 434D, and the flange 113 is clipped between the flange forward-side claw portions 434B and the flange rear-side claw portions 434A. Since the flange forward-side claw portions 434B protrude to the inside during this process, they are moved to the forward side of the flange 113 while deforming outward at the points of joint to the annular member 431, under resisting forces due to their contact with the flange 113, and they return into their original shapes on the forward side of the flange 113, whereon the claws are caught on the flange 113. After the catch of the claws on the flange 113 is confirmed, the fingers are taken off the pressing members 438A and 438B, resulting in a state in which the protruding portions 432A and 432B are in contact with the blade members 125A and 125B, as shown in FIG. 22(B). It is to be noted that the mounting of the mounting fixture 430 may be performed, not after suction of the liquid via a flow port 111 by the plunger 120 but before the suction.

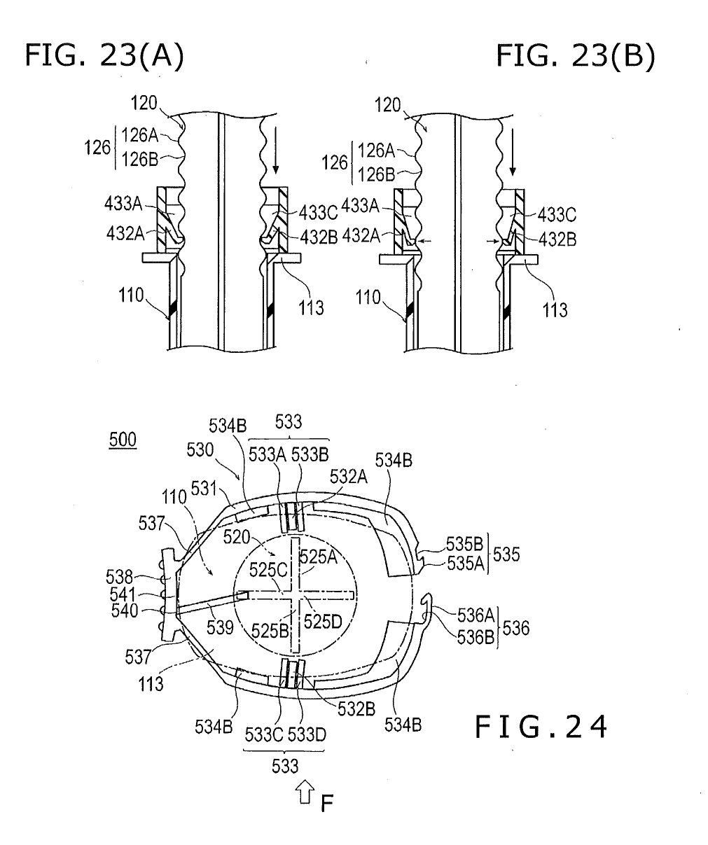

Then, when the operating member 123 is pushed forward to translate the plunger 120 in order to deliver the liquid, the protruding portions 432A and 432B making contact with the concave portions 126B gradually slide on the concave and convex portions 126, being deformed, as shown in FIG. 23. Then, when the protruding portions 432A and 432B contact the convex portions 126A, as shown in FIG. 23(B), loads exerted on the protruding portions 432A and 432B are maximized, so that deformations of the protruding portions 432A and 432B are maximized. In this instance, resisting forces act on the plunger 120, as well. Since the shifting of the plunger 120 in a rotating direction is restrained by the guide members 433A to 433D and the two protruding portions 432A and 432B are so provided as to clip the plunger 120, however, positional balancing is offered by resisting forces in opposite directions that are exerted from the two protruding portions 432A and 432B opposite each other, so that lateral shifting (shifting in a direction crossing the longitudinal direction) is restrained, as well. When the plunger 120 is advanced further, the protruding portions 432A and 432B each advance to the next concave portion 126B, whereby the loads on the protruding portions 432A and 432B are weakened, so that the protruding portions 432A and 432B return into their original shapes. In this instance, a target unit amount of liquid is delivered through the flow port 111; in addition, at the operating member 123, a variation in the pressing force (pressure) is transmitted to the finger. Therefore, the fact that the liquid has been delivered can be recognized through a sensation in the finger, without the need to visually check graduations 114. Thereafter, the operating member 123 is pushed further, as required, whereby the protruding portions 432A and 432B each slide while sequentially contacting the concave portion 126B and the convex portion 126A in this order. Accordingly, a target unit amount of liquid can be delivered through the flow port 111, while recognizing the concave portion 126B and the convex portion 126A through a sensation in the finger.

Thus, the syringe 400 according to the fourth embodiment is provided with: the external cylinder 110 fillable with a liquid therein; the plunger 120 translatable within the external cylinder 110 along the longitudinal direction of the external cylinder 110 and formed with the plurality of concave portions 126B and convex portions 126A aligned in the longitudinal direction; and the mounting fixture 430 having the annular member 431 in a ring shape that is mountable to the external cylinder 110 by inserting the plunger 120 therein from the rear end side of the plunger 120. Therefore, the mounting fixture 430 can be mounted to the external cylinder 110 from the rear end side of the plunger 120. Accordingly, the mounting fixture 430 can be easily mounted in a short time, and can be mounted selectively only when necessary. In addition, the mounting fixture 430 is provided with the protruding portions 432A and 432B each making contact sequentially with the plurality of concave portions 126B and convex portions 126A in a slidable manner as the plunger 120 is translated, and the rotation limiting member 433 contacting the plunger 120 to limit a rotation of the plunger 120 relative to the external cylinder 110. Therefore, notwithstanding the mounting fixture 430 is of a selectively mounted type, rotation of the plunger 120 is limited by the rotation limiting member 433, whereby the contact of the protruding portions 432A and 432B with the concave portions 126B and the convex portions 126A is secured reliably, and, under this condition, the protruding portions 432A and 432B contact the concave portions 126B and the convex portions 126A of the plunger 120 sequentially. Accordingly, resisting forces exerted on the plunger 120 from the protruding portions 432A and 432B are transmitted to the finger, so that the fact that a target unit amount of liquid has been delivered can be recognized through a sensation in the finger.

In addition, the annular member 431 has the pair of pressing members 438A and 438B located opposite to each other, and the pair of translating members 439A and 439B formed between the pair of pressing members 438A and 438B and translated away from each other by pressing the pair of pressing members 438A and 438B so as to bring them toward each other. Therefore, the mounting fixture 430 can be mounted from the rear end side of the plunger 120, in a state in which the pair of translating members 439A and 439B are spaced from each other by pressing the pressing members 438A and 438B. Accordingly, the mounting is facilitated, and operability is enhanced.

Furthermore, the protruding portions 432A and 432B are formed to extend from the translating members 439A and 439B toward the plunger 120. Therefore, the protruding portions 432A and 432B can be moved away from the plunger 120, together with the translating members 439A and 439B at the time of mounting. Accordingly, the mounting is facilitated, and operability is enhanced.

In addition, the rotation limiting member 433 is formed to extend from the translating members 439A and 439B toward the plunger 120. Therefore, the rotation limiting member 433 can be moved away from the plunger 120, together with the translating members 439A and 439B at the time of mounting. Accordingly, the mounting is facilitated, and operability is enhanced.

Furthermore, the mounting fixture 430 has the flange-engaging members 434 engaging the flange 113 at the edge of an opening portion 112 of the external cylinder 110 in which the plunger 120 is inserted. Therefore, the mounting fixture 430 can resist forces exerted thereon from the plunger 120 due to forward and rearward translations of the plunger 120. Consequently, the mounting fixture 430 would not easily be disengaged from the external cylinder 110, and a stable liquid delivery operation can be achieved.

In addition, the annular member 431 has the deformable portion 437 lower in flexural rigidity than other portions of the annular member 431. Therefore, the annular member 431 can be easily deformed so as to allow easy insertion of the plunger 120 therein. Accordingly, operability is enhanced.

It is to be noted that combinations of the protruding portions 432A and 432B and the blade members 125A to 125D are not restricted to the aforementioned forms, and the combinations can be changed according to the use or the like. For instance, as has been described in the first embodiment and the second embodiment, there may be adopted a configuration in which only one protruding portion is provided, all of four blade members are formed with concave and convex portions, and the blade members have different pitches of the concave portions and convex portions. In this case, the blade member to be brought into contact with the protruding portion can be selected from the four blade members, whereby the unit amount by which the liquid can be delivered can be selected from four choices.

Furthermore, the numbers and shapes of the flange forward-side claw portions 434B, the flange rear-side claw portions 434A and the guide members 433A to 433D can be changed, as required, according to the use or the like.

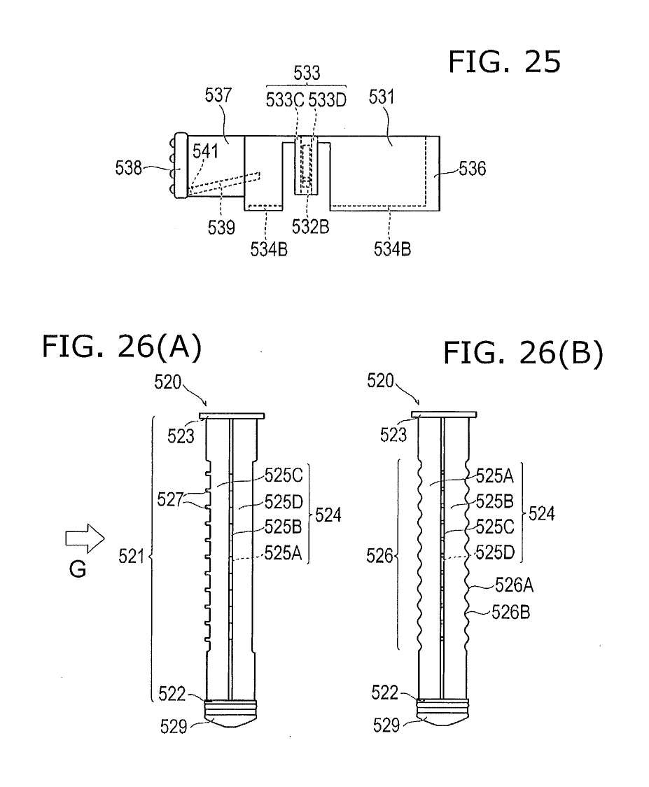

As shown in FIGS. 24 and 25, a syringe 500 according to a fifth embodiment includes: an external cylinder 110; a plunger 520 translatable within the external cylinder 110 along the longitudinal direction of the external cylinder 110; and a mounting fixture 530 mountable to the external cylinder 110. It is to be noted that since the external cylinder 110 is the same as in the first embodiment above, it is denoted by the same reference sign as used above and description thereof is omitted.

As shown in FIG. 26, the plunger 520 is provided with a plunger main body 521 extending in the longitudinal direction of the external cylinder 110, and a gasket 529 interlocked to the forward end side of the plunger main body 521. The plunger main body 521 includes: a plunger forward end 522 which is provided on the forward side and to which the gasket 529 is interlocked; an operating member 523 provided on the rear side and pushed or pulled by an operator's finger or fingers; and a plunger extension portion 524 extending long from the operating member 523 to the plunger forward end 522. The plunger extension portion 524 is provided with four plate form blade members 525A to 525D aligned about the axis of the external cylinder 110.

Out of the four blade members 525A to 525D, the two blade members 525A and 525B (concave and convex portion-forming members) located on opposite sides of the axis of the external cylinder 110 are each formed with a concave and convex portion 526 aligned in the longitudinal direction. The concave and convex portion 526 has repeating structures aligned at regular intervals, each of the repeating structures including a convex portion 526A and a concave portion 526B.

The blade member 525C (stopper-forming member) has a straight shaped part formed with convex stoppers 527 which periodically protrude at regular intervals. Another blade member 525D has a longitudinally straight shape, without any particular concave and/or convex shape formed thereon. It is to be noted that the shape of the concave and convex portion 526 is the same as the shape of the concave and convex portion 126 in the first embodiment. The pitch of the convex portions 526A is equal to the pitch of the convex stoppers 527; however, one of the pitches may be an integer times the other, or the two pitches may be quite different from each other. The plunger main body 521 is not specifically restricted in regard of material; for example, the aforementioned materials applicable to the external cylinder 110 can be used.

As shown in FIGS. 24 and 25, the mounting fixture 530 includes: an annular member 531 in a discontinuous ring shape that is mountable to the external cylinder 110 in the direction crossing the longitudinal direction so as to surround and clip a flange 113 of the external cylinder 110; two protruding portions 532A and 532B contacting the concave and convex portions 526 of the plunger 520 inserted in the external cylinder 110; a rotation limiting member 533 limiting a rotation of the plunger 520 relative to the external cylinder 110; and a flange-engaging member 534 engaging the flange 113.

The mounting fixture 530 further includes: a pair of pressing members 538 for pressing toward the inside of the ring shape by fingers; a protruding stopper 539 (protruding portion) protruding from the pressing member 538 toward the inside; two pressing member supports 537 (deformable portions) linked to the pressing member 538 in left-right symmetry; and a first interlock member 535 and a second interlock member 536 which are provided at two discontinuous ends of the annular member 531 and which are interlockable to each other.

The pressing member 538 is located on a shorter edge side of the flange 113 which is roughly rectangular in shape. Therefore, the pressing member 538 can allow for a certain length of the protruding stopper 539 extending from the pressing member 538 toward the blade member 525C. It is to be noted that the pressing member 538 may be disposed on the longer edge side of the flange 113, if the pressing member 538 can allow for the length of the protruding stopper 539 extending from the pressing member 538 toward the blade member 525C.

The pressing member supports 537 are so formed as to interpose the pressing member 538 therebetween, and are thinner, and hence lower in flexural rigidity and more flexible, than the pressing member 538. This facilitates an operation of pressing the pressing member 538, and ensures that the annular member 531 is opened wider than the distance between adjacent tips of the blade members 525A to 525D of the plunger 520 at the time of mounting with the pressing member support 537 as a fulcrum, so that the plunger 520 and the external cylinder 110 can be accommodated into the inside of the annular member 531.

The protruding stopper 539 contacts the flange 113 at a joint to the annular member 531, and is protruding toward the plunger 520. The protruding stopper 539 protrudes from a position 540 deviated from the center 541 of the pressing member 538 along the circumferential direction of the ring shape, and its tip is located so as to contact the blade member 525C, which is formed with the convex stoppers 539 in front of the center 541 of the pressing member 538. In other words, the protruding stopper 539 is protruding so as to contact the blade member 525C from an oblique direction, as viewed from the rear side as shown in FIG. 24. Further, as shown in

FIG. 25, the protruding stopper 539 is formed to protrude obliquely toward the rear side. The protruding stopper 539 is provided with a cut 541 in its base portion, whereby it is made thinner there, so that it is pliable toward the forward side and the rear side.

As shown in FIGS. 24 and 25, the protruding portions 532A and 532B are formed on the inside of the annular member 531, are capable of contacting the concave and convex portions 526 of the plunger 520, and are retractable through deforming in the manner of elastically bending so as to come away from the plunger 520. Therefore, each time the plunger 520 is advanced or retracted within the external cylinder 110, the protruding portions 532A and 532B are deformed, and make sliding contact sequentially with the plurality of convex portions 526A and concave portions 526B. It is to be noted that there may be adopted a structure in which the protruding portions 532A and 532B themselves are not bent, and the annular member 531 provided with the protruding portions 532A and 532B is deformable in a direction away from the plunger 520.

The rotation limiting member 533 is provided with four guide members 533A to 533D formed on the inside of the annular member 531. In a state in which the mounting fixture 530 is mounted to the external cylinder 110, the two guide members 533A and 533B are disposed in parallel, with a predetermined spacing therebetween, so as to clip the blade member 525A of the plunger 520 from a direction perpendicular to the longitudinal direction of the external cylinder 110. The other two guide members 533C and 533D are disposed in parallel, with a predetermined spacing therebetween, so as to clip the blade member 525B from a direction perpendicular to the longitudinal direction of the external cylinder 110. When the plunger 520 is advanced or retracted within the external cylinder 110, therefore, the guide members 533A to 533D function as walls which limit a rotation of the plunger 520 relative to the external cylinder 110 and which hold the concave and convex portions 526 of the plunger 520 so as to prevent the concave and convex portions 526 from being disengaged from the protruding portions 532A and 532B through shifting in a rotating direction of the plunger 520.

The flange-engaging member 534 has flange forward-side claw portions 534B contacting a forward-side surface of the flange 113 and protruding from outside toward inside in conformity with a curved shape of the annular member 531. The flange 113 is clipped between the flange forward-side claw portions 534B and the guide members 533A to 533D, which contact a rear-side surface of the flange 113, whereby the mounting fixture 530 can be fixed to the flange 113. When the mounting fixture 530 is fixed to the flange 113 by the guide members 533A to 533D and the flange forward-side claw portions 534B, the mounting fixture 530 can resist forces exerted thereon from the plunger 520 due to forward and rearward translations of the plunger 520, so that the mounting fixture 530 would not easily be disengaged from the external cylinder 110. It is to be noted that the numbers and shapes of the guide members 533A to 533D and the flange forward-side claw portions 534B are not specifically restricted, insofar as they can stably engage the flange 113.

The first interlock member 535 and the second interlock member 536 are provided with a first claw portion 535A and a second claw portion 536A engaging each other, and a first hole portion 535B and a second hole portion 536B which allow easy bending of the first claw portion 535A and the second claw portion 536A for engagement with each other. Mutual engagement of the first claw portion 535A and the second claw portion 536A fixes the mounting fixture 530 to the flange 113 in such a manner as to surround the plunger 520.

Now, operation of the syringe 500 according to the fifth embodiment will be described below.

First, before mounting of the mounting fixture 530, the external cylinder 110 and the plunger 520 in a state in which a liquid is accommodated in the external cylinder 110 and the plunger 520 is located on the rear side in the inside of the external cylinder 110, in other words, in a state in which the liquid can be discharged via a flow port 111 by translating the plunger 520 forward, are prepared, like in the first embodiment.

Next, in order to mount the mounting fixture 530, portions near the first interlock member 535 and the second interlock member 536 are pulled and the annular member 531 is deformed with the pressing member support 537 as a fulcrum, so as to space the first interlock member 535 and the second interlock member 536 apart from each other, thereby resulting in a state in which the plunger 520 and the external cylinder 110 can be accommodated into the inside of the annular member 531.

Subsequently, the blade member 525A is located between the two guide members 533A and 533B, and the blade member 525B is located between the two guide members 533C and 533D. It follows that the protruding portion 532A is opposed to the blade member 525A, whereas the protruding portion 532B is opposed to the blade member 525B, and the protruding stopper 539 is located opposite the blade member 525C.

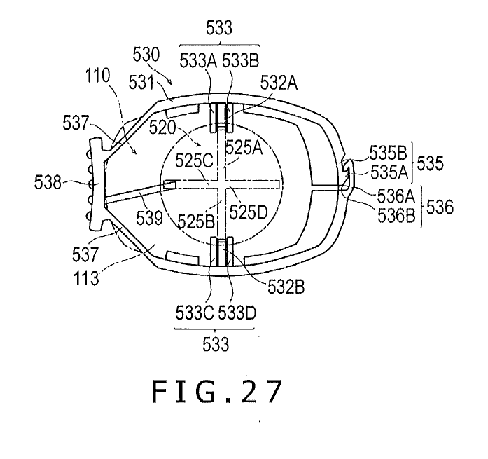

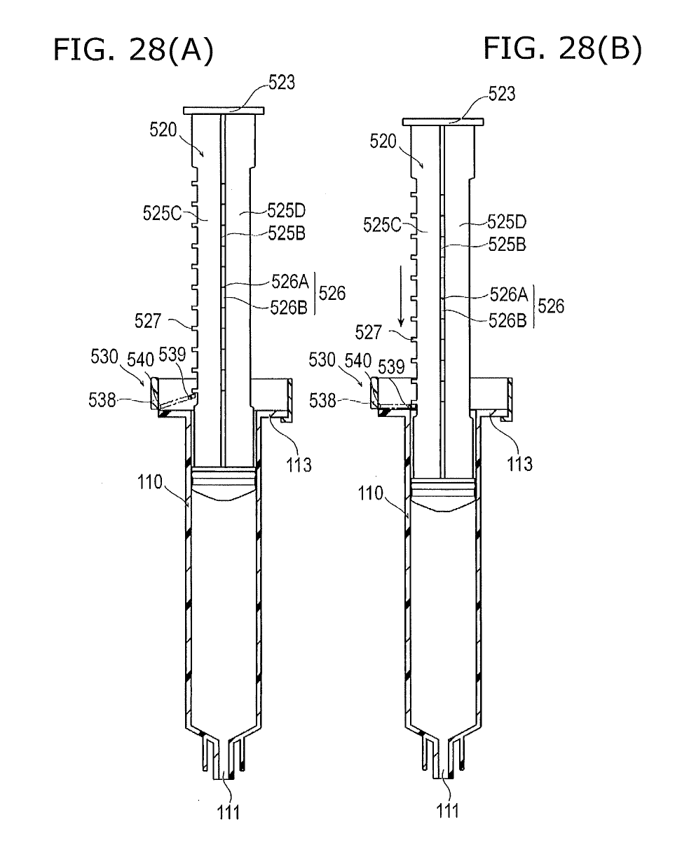

Next, the forces with which the portions of the annular member 531 are pulled are weakened, allowing the annular member 531 to return gradually into its original state. In this instance, such a positioning is conducted that the flange 113 is clipped between the guide members 533A to 533D and the flange forward-side claw portions 534B. Then, the first claw portion 535A and the second claw portion 536A are mated and engaged with each other. It follows that, as shown in FIG. 27 and FIG. 28(A), the mounting fixture 530 is fixed to the external cylinder 110 in the state of wholly accommodating the plunger 520 so as to surround the plunger 520. In this state, the blade members 525A and 525B are clipped between the guide members 533A to 533D, and the protruding portions 532A and 532B abut on the concave portions 526B of the blade members 525A and 525B. Then, the protruding stopper 539 is disposed in such a position as to be able to contact the convex stoppers 527 of the blade member 525C, by a forward translation of the plunger 520.

Then, when the operating member 523 of the plunger 520 is pushed forward to translate the plunger 520 in order to deliver the liquid, the protruding stopper 539 comes into contact with the convex stopper 527 of the blade member 525C. When the plunger 520 is translated further, an external force in a forward direction is exerted on the protruding stopper 539 by the convex stopper 527, as shown in FIG. 28(B). It follows that the protruding stopper 539 is bent with the cut 541 (see FIG. 25) as a fulcrum, while the protruding stopper 539 is being moved together with the convex stopper 527. Upon contact of the tip of the protruding stopper 539 with the flange 113, the protruding stopper 539 enters a state of extending straight along the surface of the flange 113, whereon a further translation forward is limited by the flange 113, resulting in a full stop. In this instance, a target unit amount of liquid has just been delivered through a flow port 111. Therefore, delivery of the liquid in an amount more than necessary can be restrained.

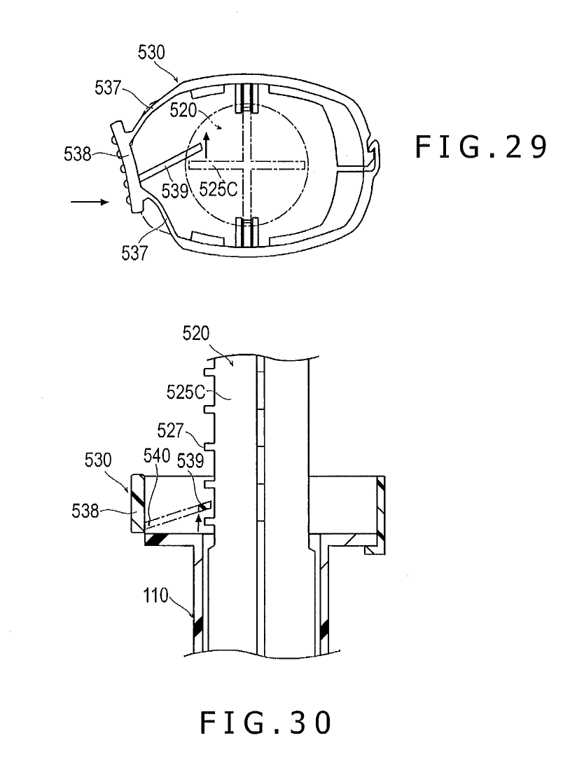

At the time of delivering the next unit amount of liquid, the side of the pressing member 538 on which the protruding stopper 539 is fixed is pressed, as shown in FIG. 29. By this operation, the pressing supports 537 are bent, and the tip of the protruding stopper 539 is moved so as to deviate circumferentially from the blade member 525C, so that locking is canceled. As a result, the protruding stopper 539 returns elastically into its original shape, moving toward the rear side, as shown in FIG. 30. Then, when the pressing on the pressing member 538 is stopped, the protruding stopper 539 is disposed on the rear side of the convex stopper 527 with which it has been engaged. As a result, a state in which the plunger 520 can be pushed in is re-established. It is to be noted that the position at which to press the pressing member 538 is not specifically restricted, insofar as the protruding stopper 539 can be deviated from the convex stopper 527 by the pressing.

Furthermore, in the operation of pushing in the plunger 520, variations in a pressing force (pressure) can also be recognized through sensation. Specifically, when the operating member 523 of the plunger 520 is pushed forward to translate the plunger 520, as shown in FIG. 28, the protruding portions 532A and 532B (see FIG. 27) making contact with the concave portions 526B deform while sliding on the concave and convex portions 526 gradually. Then, when the protruding portions 532A and 532B each contact the convex portion 526A, loads exerted on the protruding portions 532A and 532B are maximized, so that deformations of the protruding portions 532A and 532B are maximized. In this instance, resisting forces act on the plunger 520, as well. Since the shifting of the plunger 520 in a rotating direction is restrained by the guide members 533A to 533D and the two protruding portions 532A and 532B are so provided as to clip the plunger 520, however, positional balancing is offered by resisting forces in opposite directions that are exerted from the two protruding portions 532A and 532B opposite each other, so that lateral shifting (shifting in a direction crossing the longitudinal direction) is restrained, as well. When the plunger 520 is advanced further, the protruding portions 532A and 532B each advance to the next concave portion 526B, whereby the loads on the protruding portions 532A and 532B are weakened, so that the protruding portions 532A and 532B return into their original shapes. In this instance, a target unit amount of liquid is delivered through the flow port 111; in addition, at the operating member 523, a variation in the pressing force (pressure) is transmitted to the finger. Therefore, the fact that the liquid has been delivered can be recognized through a sensation in the finger, without the need to visually check graduations 114. Thereafter, the operating member 523 is pushed further, as required, whereby the protruding portions 532A and 532B each slide while sequentially contacting the concave portion 526B and the convex portion 526A in this order. Accordingly, a target unit amount of liquid can be delivered through the flow port 111, while recognizing the concave portion 526B and the convex portion 526A through a sensation in the finger.

Thus, the syringe 500 according to the fifth embodiment includes: the external cylinder 110 fillable with a liquid therein; the plunger 520 translatable within the external cylinder 110 along the longitudinal direction of the external cylinder 110; and the mounting fixture 530 mountable to the external cylinder 110. Therefore, the mounting fixture 530 can be easily mounted, selectively only when necessary. In addition, the mounting fixture 530 is provided with: the protruding stopper 539 which contacts the plunger 520 as the plunger 520 is translated, so as to stop the translation of the plunger 520; and the rotation limiting member 533 which contacts the plunger 520 so as to limit a rotation of the plunger 520 relative to the external cylinder 110. Therefore, notwithstanding the mounting fixture 530 is of a selectively mounted type, rotation of the plunger 520 is limited by the rotation limiting member 533, whereby the contact of the protruding stopper 539 with the plunger 520 is secured reliably, and the plunger 520 is thereby stopped assuredly. Accordingly, delivery of the liquid in an amount more than necessary can be restrained securely, and a target amount of the liquid can be delivered easily.

In addition, the plunger 520 has the convex stoppers 527 which are aligned in plurality in the longitudinal direction and which can contact the protruding stopper 539. Furthermore, the mounting fixture 530 has the pressing member 538 configured to fix the protruding stopper 539. With the pressing member 538 pressed, the protruding stopper 539 is moved from a position of contact with the convex stopper 527 along the circumferential direction of the plunger 520 so as to cancel holding the plunger 520. Therefore, holding the plunger 520 can be easily canceled by an operation of pressing the pressing member 538. Consequently, operability is enhanced.

Furthermore, the mounting fixture 530 has the pressing member supports 537 which are adjacent to the pressing member 538 and are lower in flexural rigidity than the pressing member 538. Therefore, the pressing member 538 can be easily pressed in, and operability is enhanced.

In addition, the mounting fixture 530 has the flange-engaging member 534 engaging the flange 113 provide at the edge of an opening portion of the external cylinder 110 in which the plunger 520 is inserted. Therefore, the mounting fixture 530 can resist forces exerted thereon from the plunger 520 due to forward and rearward translations of the plunger 520. Accordingly, the mounting fixture 530 would not easily be disengaged from the external cylinder 110, and a stable liquid delivery operation can be achieved.

It is to be noted that the protruding portions 532A and 532B may not necessarily be provided. In addition, two or more (e.g., four) blade members (stopper-forming members) may be provided with convex stoppers, at different pitches. In this case, the blade member to be brought into contact with the protruding stopper 539 can be selected from the four blade members, and the unit amount of liquid delivered can be selected from four choices.

Furthermore, in order to allow easy deviation of the protruding stopper 539 from the blade member 525C, a structure may be adopted in which that surface of the blade member 525C which is formed with the convex stoppers 527 is formed obliquely so that the protruding stopper 539 is deviated in the manner of sliding along the slant surface by being pressed in.

It is to be noted that the present invention is not limited only to the aforementioned embodiments, and various modifications can be made by those skilled in the art, within the scope of the technical thought of the invention. For example, although it is preferable that the protruding portion makes contact sequentially with the plurality of convex portions and concave portions of the plunger in a slidable manner, the protruding portion may not necessarily slide without parting from the convex portions and concave portions at all. For instance, a structure in which the protruding portion does not contact the concave portions is also included. Accordingly, it is preferable for the protruding portion to make contact sequentially with at least a plurality of convex portions in a slidable manner.

In addition, the configuration contained in each of the aforementioned embodiments may be applied to other embodiments. For instance, the protruding stopper described in the fifth embodiment may be applied to a mounting fixture in a ring shape made from a flat plate as described in the third embodiment, or to a mounting fixture in a form of being mounted on a plunger from the rear end side of the plunger as described in the fourth embodiment. Furthermore, naturally, the protruding stopper can be applied to the mounting fixture of the first embodiment or the second embodiment. In addition, a mounting fixture in a discontinuous ring shape as in the first to third and fifth embodiments can be made to be mountable in the manner of mounting it on a plunger from the rear end side of the plunger as in the fourth embodiment.

Furthermore, while the mounting fixtures in the first to fifth embodiments each engage the outside of the flange 113 of the external cylinder 110, there may be adopted, for example, a structure in which an engaging member extending from the mounting fixture to the inside of the external cylinder 110 is formed and this engaging member engages the inside of the flange 113 (external cylinder 110). A plurality of such engaging members, for example, are formed in the circumferential direction so as to contact the inside surface of the external cylinder 110, are elastically deformed by being put into the inside of the external cylinder 110, and are firmly engaged with the inside surface of the external tube 110 by repellent elastic forces. Such engaging members are especially effective in the case where a ring shape protruding portion for restraining dropping-off of the plunger 120 is formed at the inner edge of the flange 113, but a configuration may be adopted in which such a protruding portion is not formed and the engagement is achieved by only the elastic forces of the engaging members.

The detailed description above describes a syringe and mounting fixture. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A syringe comprising:
an external cylinder which is fillable with a liquid;
a plunger which is translatable within the external cylinder along a longitudinal direction of the external cylinder, the plunger formed with a plurality of concave portions and convex portions aligned in the longitudinal direction; and
a mounting fixture that includes a ring-shaped annular member mountable to the external cylinder, a protruding portion configured to, upon translation of the plunger within the external cylinder, contact at least the plurality of convex portions sequentially in a slidable manner or stop movement of the convex portions, and a rotation limiting member contacting the plunger so as to limit a rotation of the plunger relative to the external cylinder.

2. The syringe according to claim 1, wherein the annular member is formed in a discontinuous ring shape mountable to the external cylinder by being mounted on the external cylinder or the plunger from a direction crossing the longitudinal direction of the external cylinder.

3. The syringe according to claim 2, wherein the mounting fixture has a first interlock member and a second interlock member interlockable to each other and located at respective discontinuous ends.

4. The syringe according to claim 1, wherein the rotation limiting member has one more pairs of guide members configured to clip the plunger from a direction perpendicular to the longitudinal direction of the external cylinder.

5. The syringe according to claim 4, wherein the one or more pairs of guide members are closely aligned so as to clip the plunger from a spaced relationship with each other, through deformation of the mounting fixture when the mounting fixture is mounted.

6. The syringe according to claim 1, wherein the mounting fixture has a flange-engaging member engaging a flange at an edge of an opening portion of the external cylinder in which the plunger is inserted.

7. The syringe according to claim 6, wherein the mounting fixture includes: a rear-side main body disposed in a side of the opening portion of the flange of the external cylinder; a forward-side main body disposed opposite the side of the opening portion of the flange; a connecting member connecting the forward-side main body and the rear-side main body; and a main body engaging member capable of interlocking the forward-side main body and the rear-side main body.

8. The syringe according to claim 2, wherein the annular member is a flat-shaped member in a flat plate form that is mountable to the external cylinder by deformation into a ring shape.

9. The syringe according to claim 1, wherein the annular member is formed in a ring shape so as to be mountable to the external cylinder by inserting the plunger therein from a rear end side of the plunger.

10. The syringe according to claim 9,
wherein the annular member includes:
a pair of pressing members located opposite each other; and
a pair of translating members formed between the pair of pressing members and configured to translate so as to be spaced from each other by pressing the pair of pressing members closer to each other.

11. The syringe according to claim 1, wherein the protruding portion is a protruding stopper configured to stop a translation of the plunger by contacting the plunger as the plunger translates.

12. The syringe according to claim 11,
wherein the convex portion is a convex stopper that is capable of contacting the protruding stopper,
the mounting fixture has a pressing member configured to fix the protruding stopper, and
the protruding stopper cancels holding the plunger by moving in a circumferential direction of the plunger from a position of contact with the convex stopper when the pressing member is pressed.

13. The syringe according to claim 1,
wherein the plunger has a plurality of concave and convex portion-forming members each of which has the plurality of concave portions and convex portions aligned in the longitudinal direction of the plunger and which are aligned about an axis line of the plunger, and
the pitch of the concave portions and convex portions differs depending on the concave and convex portion-forming members.

14. The syringe according to claim 2, wherein the rotation limiting member has one more pairs of guide members configured to clip the plunger from a direction perpendicular to the longitudinal direction of the external cylinder.

15. The syringe according to claim 3, wherein the rotation limiting member has one more pairs of guide members configured to clip the plunger from a direction perpendicular to the longitudinal direction of the external cylinder.

16. A mounting fixture comprising:
a ring-shaped annular member mountable to an external cylinder;
a protruding portion configured to, upon translation of a plunger within the external cylinder, contact at least a plurality of convex portions formed on the plunger sequentially in a slidable manner or stop movement of the convex portions; and
a rotation limiting member configured to contact the plunger so as to limit a rotation of the plunger relative to the external cylinder.

17. The mounting fixture according to claim 16, wherein the annular member is formed in a discontinuous ring shape.

18. The mounting fixture according to claim 16, further comprising a first interlock member and a second interlock member interlockable to each other and located at respective discontinuous ends.

19. The mounting fixture according to claim 16, wherein the rotation limiting member has one more pairs of guide members configured to clip the plunger from a direction perpendicular to a longitudinal direction of the external cylinder.

20. The mounting fixture according to claim 16,
wherein the annular member includes:
a pair of pressing members located opposite each other; and
a pair of translating members formed between the pair of pressing members and configured to translate so as to be spaced from each other by pressing the pair of pressing members closer to each other.
